SEQUENCE LISTING (3 sequences)

SEQ ID NO: 1 - protein, 1675 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 2 - protein, 248 aa, Homo sapiens
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVH

SEQ ID NO: 3 - protein, 229 aa, Homo sapiens
MADDFGFFSSSESGAPEAAEEDPAAAFLAQQESEIAGIENDEGFGAPAGSHAAPAQPGPTSGAGSEDMGTTVNGDVFQEANGPADGYAAIAQADRLTQEPESIRKWREEQRKRLQELDAASKVTEQEWREKAKKDLEEWNQRQSEQVEKNKINNRIADKAFYQQPDADIIGYVASEEAFVKESKEETPGTEWEKVAQLCDFNPKSSKQCKDVSRLRSVLMSLKQTPLSR

(19) United States
(12) Patent Application Publication
(10) Pub. No.: US 2023/0293722 A1
(43) Pub. Date: Sep. 21, 2023

(54) Compositions and Methods for Treating Neurological-associated Disorders

(71) Applicant: MetaQor LLC, Boston, MA (US)

(72) Inventors: Franco Vitaliano, Boston, MA (US); Gordana Vitaliano, Boston, MA (US)

(21) Appl. No.: 18/093,775

(22) Filed: Jan. 5, 2023

Related U.S. Application Data
(60) Provisional application No. 63/296,666, filed on Jan. 5, 2022.

(51) Int. Cl. A61K 47/69; A61K 9/51; A61P 31/18; A61P 25/28; A61K 47/68; A61K 31/496; A61K 31/195; A61K 31/137; A61K 31/7076; A61K 31/4458; A61K 31/437; A61K 31/55; A61K 31/165; A61K 31/485; A61K 31/27; A61K 31/357; A61K 49/08

(52) U.S. Cl. CPC A61K 47/6931; A61K 9/5169; A61P 31/18; A61P 25/28; A61K 47/6891; A61K 31/496; A61K 31/195; A61K 31/137; A61K 31/7076; A61K 31/4458; A61K 31/437; A61K 31/55; A61K 31/165; A61K 31/485; A61K 31/27; A61K 31/357; A61K 47/6801; A61K 49/085; A61K 49/08

(57) ABSTRACT

Compositions comprising clathrin nanoparticles and methods for treating neurological-associated disorders.

### CROSS-REFERENCE TO RELATED APPLICATIONS

This application claims the benefit of priority to U.S. application Ser. No. 63/296,666, filed on Jan. 5, 2022, the contents of which are hereby incorporated by reference.

### FEDERALLY SPONSORED RESEARCH OR DEVELOPMENT

This invention was made with Government support under Grant No. MH108481-04 awarded by the National Institutes of Health. The Government has certain rights in the invention.

### SEQUENCE LISTING

This application contains a Sequence Listing that has been submitted electronically as an XML file named “41460-0006001_SL_ST26.XML.” The XML file, created on May 24, 2023, is 5,744 bytes in size. The material in the XML file is hereby incorporated by reference in its entirety.

### TECHNICAL FIELD

The present invention relates to compositions comprising clathrin nanoparticles and methods for treating neurological-associated disorders.

### BACKGROUND

Neurotrophic factor (NTF) biologics have been studied as treatments for neurodegenerative disorders (e.g., Alzheimer's disease, HIV-Associated Neurocognitive Disorder (HAND), Parkinson's, and Huntington's diseases), neuroinflammatory diseases (e.g., amyotrophic lateral sclerosis and multiple sclerosis), stroke, traumatic brain injury, and psychiatric disorders (e.g., depression). One of the most studied NTFs is brain-derived neurotrophic factor (BDNF). BDNF is widely distributed in the CNS, and it plays a central role in cell metabolism, survival, growth, and maintenance. Its ability to modulate synaptic plasticity and learning and memory processes, as well as its protective effects in adult brain, have been documented. Early intervention with BDNF treatment can help prevent or impede the development of neurodegenerative disorders. However, BDNF's properties, including its charge, extremely short plasma half-life on the order of minutes, poor pharmacokinetic profile, and poor blood-brain barrier (BBB) penetrability, limit its use as a therapeutic. Thus, parenteral or intranasal BDNF administration results in minimal CNS delivery.

Different strategies have been developed to either deliver BDNF into the brain or to increase its CNS concentrations including: 1) direct CNS infusions, 2) cell and gene therapy via injection of stem cells or viral vectors, 3) polymer implants that release BDNF, and 4) ultrasound enhanced BDNF delivery. However, these methods require invasive procedures that are not suitable for routine clinical practice. Noninvasive BDNF delivery methods using nanoparticles (NPs) capable of crossing the nasal barrier or the BBB have achieved varying degree of success. For example, BDNF was transported across the BBB via anti-transferrin receptor antibodies or poloxamor-188 polymeric NPs. Also, BDNF encapsulated in polymer, or fused with cell-penetrating peptides and packaged in adenovirus associated virus NPs passed the nasal barrier. However, modifications that increase NP stability and/or CNS penetrability often also increase immunogenicity or toxicity. Therefore, there is a strong need for improved therapies for treating neurodegenerative disorders (e.g., Alzheimer's disease, HIV-Associated Neurocognitive Disorder, Parkinson's, and Huntington's diseases), neuroinflammatory diseases (e.g., amyotrophic lateral sclerosis and multiple sclerosis), stroke, traumatic brain injury, and psychiatric disorders (e.g., depression).

### SUMMARY

Provided herein are compositions comprising: (i) a CNS targeting agent; and (ii) a neurotrophic factor, wherein the CNS targeting agent and the neurotrophic factor are linked to a clathrin nanoparticle. Also provided herein are methods for treating a human subject having or at risk for developing a neurodegenerative disorder, optionally Alzheimer's disease, HIV-Associated Neurocognitive Disorder (HAND), Parkinson's disease, or Huntington's diseases; a neuroinflammatory disease, optionally amyotrophic lateral sclerosis or multiple sclerosis; stroke; a neurological disorder associated with COVID-19, optionally a cognitive deficit; traumatic brain injury; or a psychiatric disorder, optionally depression, comprising administering to the human subject a composition comprising: (i) a CNS targeting agent; and (ii) a neurotrophic factor, wherein the CNS targeting agent and the neurotrophic factor are linked to a clathrin nanoparticle.

In some embodiments, the clathrin nanoparticle comprises a clathrin cage.

In some embodiments, the clathrin nanoparticle consists of a clathrin triskelion.

In some embodiments, the clathrin nanoparticle comprises a complex consisting of 1 to 3 clathrin heavy chains.

In some embodiments, the neurotrophic factor is a neurotrophic factor that binds to tropomyosin-related kinase receptor, optionally TrkA, TrkB, and/or TrkC, NT-3 receptor, NT-4 receptor, NT-5 receptor, neurturin receptor, persephin receptor, artemin receptor, ciliary neurotrophic factor receptor, p75 neurotrophin receptor, or leukemia inhibitory factor receptor.

In some embodiments, the neurotrophic factor is linked to the clathrin nanoparticles by conjugation.

In some embodiments, the neurotrophic factor is conjugated to the clathrin nanoparticles via PEG.

In some embodiments, a clathrin heavy chain of the clathrin triskelion is linked to 1 to 5 molecules of the neurotrophic factor.

In some embodiments, the clathrin nanoparticle comprises a complex consisting of 3 clathrin heavy chains.

In some embodiments, each of the clathrin heavy chains is linked to 1 to 5 molecules of the neurotrophic factor.

In some embodiments, each of the clathrin heavy chains is linked to 1 molecule of the neurotrophic factor.

In some embodiments, the neurotrophic factor is NT-3, NT-4, NT-5, NT-6, neurturin, persephin, artemin, ciliary neurotrophic factor, nerve growth factor (NGF), or leukemia inhibitory factor.

In some embodiments, the CNS targeting agent is an antibody.

In some embodiments, the antibody comprises an anti-CD11b antibody, an anti-TrkB receptor antibody, an anti-DAT antibody, an anti-GABA antibody, an anti-SYP antibody, an anti-serotonin transporter antibody, an anti-dopamine-1 antibody, an anti-dopamine-2 antibody, an anti-dopamine-3 antibody, an anti-TREM2-antibody, an IL-6R-antibody, or an anti-TNF-α-antibody.

In some embodiments, the antibody is a monoclonal antibody.

In some embodiments, the CNS targeting agent is a ligand.

In some embodiments, the ligand comprises a DAT ligand, a TSPO ligand, a GABA ligand, a serotonin transporter ligand, a D1 ligand, a D2 ligand, or a D3 ligand.

In some embodiments, the composition as described above in any of the foregoing embodiments further comprises an imaging contrast agent.

In some embodiments, the imaging contrast agent comprises a gadolinium (Gd) based contrast agent, manganese-based, superparamagnetic iron oxide (SPIO), monocrystalline iron oxide nanoparticle (MION), cross-linked iron oxide (CLIO), or magneto-dendrimer contrast agent.

In some embodiments, the composition as described above in any of the foregoing embodiments further comprises one or more additional therapeutic agent.

In some embodiments, the additional therapeutic agent comprises aripiprazole, baclofen, bupropion, d-AMP and MPH-SR, dextroamphetamine, gabapentin, ibudilast, methylphenidate, mirtazapine, modafinil, NAC, naltrexone, rivastigmine, or topiramate.

In some embodiments, the composition as described above in any of the foregoing embodiments comprises the neurotrophic agent, targeting agent, imaging contrast agent and/or additional therapeutic agent linked together with the clathrin nanoparticle.

In some embodiments, the HIV-Associated Neurocognitive Disorder (HAND) is asymptomatic neurocognitive impairment (ANI), mild neurocognitive disorder (MND), or HIV associated dementia (HAD).

In some embodiments, the method further comprises imaging a region of the brain using the contrast agent during course of treating the neurodegenerative disorder.

In some embodiments, the composition is delivered intranasally or intravenously.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 70% (e.g., at least 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the Needleman and Wunsch ((1970) J. Mol. Biol. 48:444-453) algorithm, which has been incorporated into the GAP program in the GCG software package (available at gcg.com), using either a Blossum 62 matrix, a PAM250 matrix, a NWSgapdna.CMP matrix. In some embodiments, the percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1E. BDNF-Clathrin (BDNF-CT) nanoparticle purification and characterization. Transmission electron microscopy (TEM) images show (a) clathrin coated vesicles (CCVs) isolated from pig brains, and (b) purified clathrin triskelia (CT) with attached metal (Gd) negatively stained with 1% uranyl acetate. (c) A graphical representation of a clathrin triskelion with 3 heavy chains (CHCs, gray with blue terminal domains) and 3 light chains (LCs, red). One BDNF molecule is conjugated to the each CHC via PEG (green) and NP solution had 2 mg/ml of CT with 0.25 mg/ml of BDNF and 0.194 mg/ml of PEGs. (d) SDS-PAGE shows the CHC molecular weight increased by 29.4 kDa following BDNF conjugation to CT. (e) Dynamic light scattering (DLS) studies show the mean hydrodynamic radius (RH) of unconjugated CT to be 16.8±5.6 nm (top). The mean RH increased to 35.1±12.6 nm after BDNF PEGylation and subsequent conjugation to CT (bottom).

FIGS. 2A-2P. CT efficiently delivered BDNF to TrkB receptors in the mouse brain. Tat+ mice were given a single dose of i.n. BDNF-CT-Rho (0.3 mg/kg of BDNF & 2.4 mg/kg of CT). Brains were collected after 4 h. Brain sections were probed for TrkB and visualized using Alexa Fluor 488. Confocal microscopy shows TrkB receptors (green) (a, b) and intranasally delivered BDNF-CT-Rho (red) nanoparticles (c, d). BDNF-CT-Rho are co-localized with TrkB receptors (yellow) (e, f). Images were taken from two DG locations (box; g, h). Scale bars are 2 μm. Representative images of the BDNF-CT-Rho are shown in the frontal cortex (FC) (i), striatum (STR) (j), hippocampus (HPC) (k) and hypothalamus (HTH) (1). Brains were also collected from Tat+ mice at 2, 4 and 24 h after i.n. [3]H-BDNF-CT NP administration. Tissue concentrations of radioactive [3]H-30 BDNF-CT were assessed. The peak concentrations of BDNF-CT in the FC (m), ST (n), HPC (o) and HTH (p) were achieved 4 h after administration. Data are represented as the mean % of the injected dose per gram (% ID/g) of tissue (n=3-4/group and for 24 h n=2/group). Error bars represent S.D. Scale bars are 50 μm.

FIGS. 3A-3I. CT are required for BDNF delivery to the hippocampus and increased Akt expression and signaling. Tat+ mice received i.p. Dox and one of four i.n. treatments (BDNF, Sal, CT or BDNF-CT) daily for 4 days (a). Global control (Tat−) mice received i.p. and i.n. saline (Tat−/Sal). The hippocampal tissue shows increased mBDNF (b), proBDNF (c), mBDNF/proBDNF ratio (d), Akt (e) pAkt (f) and the pAkt/Akt ratio (g) in Tat+ mice that received BDNF-CT vs. other treatments (*p<0.05,**p<0.01 and ***p<0.001), as well as vs. saline treated Tat− mice (# p<0.05 and ## p<0.01, ### p<0.001). Tat+ mice treated with BDNF-CT or Sal had higher pAkt/Akt ratios (g) compared to Tat− controls but levels of Akt and pAkt were significantly higher in BDNF-CT vs. Sal treated Tat+ mice indicating enhanced Akt expression and signaling only with BDNF-CT NPs. Representative WB images are shown (h and i). Data are shown as the mean % change from Tat− control (Tat−/Sal) represented by dotted lines (n=4-5 per group, 5 cohorts were processed in parallel). Error bars represent S.E.M.

FIGS. 4A-4M. BDNF-CT increased newborn cell survival, proliferation and neurogenesis. Tat+ mice received i.p. Dox (100 mg/kg) and i.n. saline or BDNF-CT (0.3 mg/kg of BDNF & 2.4 mg/kg of CT) daily for 7 days. Tat− controls received i.p. and i.n. saline. All mice received BrdU (50 mg/kg, i.p. every 12 h) on days 1 and 2 and were sacrificed on the 7th day. Hippocampal sections containing the granule cell layer (GLC) were evaluated for BrdU+, Ki67+ and DCX+ cells using IHC with nickel enhanced DAB staining (black). Densities of BrdU+ cells (a), Ki67+ cells (b), and DCX+ cells (c) are higher in BDNF-CT-treated Tat+ mice vs. matched controls (Tat+ and Tat−) that received saline. Data are represented as the mean density. Error bars represent S.E.M. (n=4-6 per group, *<0.05, **p<0.01, ***p<0.001 and ****p<0.0001). Images represent BrdU+(d, e, f), Ki67+ (g, h, i) or DCX+ (j, k, 1) staining (black) in the hippocampal GCL following administration of Sal/Sal to Tat− mice, and Dox/Sal or Dox/BDNF-CT to Tat+ mice. Higher magnification of the marked area (see 1) with DCX+ cells are shown (m). Color saturation is 0% and scale bars are 50 μm.

FIGS. 5A-5K. BDNF-CT rescues neurogenesis from Tat-induced toxicity in the hippocampus. Tat+ mice received i.p. Dox (100 mg/kg) and i.n. saline or BDNF-CT nanoparticle (0.3 mg/kg of BDNF & 2.4 mg/kg of CT) daily for 7 days. Tat− controls received i.p. and i.n. saline. All mice received BrdU (50 mg/kg, i.p. every 12 h) on days 1 and 2 and were sacrificed on the 7th day (a). Hippocampal sections containing the granule cell layer were evaluated and double labeled cells (BrdU+ and DCX+) are expressed as the percentage of BrdU+ cells (b). The mean percentage was higher in BDNF-CT- treated Tat+ mice vs. matched controls (Tat+ and Tat−) that received saline, and was lower in Tat+ vs. Tat− mice. BrdU+ cells (red, c, d, e), DCX+ cells (green, f, g, h) and double labeled cells (BrdU+ and DCX+) (i, j, k) are shown. Nuclei (blue) were labeled with DAPI. Data are represented as the mean percentage. Error bars represent S.E.M. (n=3 per group; *<0.05, **p<0.01 and ***p<0.001).

FIGS. 6A-6E. BDNF-CT enhanced synaptogenesis and dendritic integrity in the Tat+ mouse hippocampus. Tat+ mice received i.p. Dox (100 mg/kg) and i.n. saline or BDNF-CT (0.23 mg/kg of BDNF & 1.85 mg/kg of CT) daily for 7 days. Cognitive testing started on the 8th day and lasted for 5 days (a). Mice were sacrificed on day 12 and hippocampal dentate gyms (DG), CA1 and CA3 sections were evaluated for SYP+ (b) (red) and MAP2+(c) (green) staining. The SYP (d) and MAP2 (e) immunoreactivities (IR) increased in the hippocampal DG, CA1 and CA3 regions in BDNF-CT treated Tat+ mice vs. saline treated controls. Data are shown as the mean % change from saline treated controls. Error bars represent S.E.M. (n=6-8 per group; *P<0.05, **P<0.01, ***P=0.001).

FIGS. 7A-7H. BDNF-CT ameliorated Tat-induced learning and memory deficits and enhanced cognitive flexibility in the Novel object recognition test (NORT) and Barnes maze test (BMT). Tat+ mice received i.p. Dox (100 mg/kg) and either i.n. saline or BDNF-CT daily for 7 days. Testing procedures started on the 8th day and lasted for 5 days. (a) NORT Phase 3 RI was significantly higher in Tat+ mice treated daily with a high dose (0.3 mg/kg of BDNF & 2.4 mg/kg of CT) but not a low dose (0.15 mg/kg of BDNF & 1.2 mg/kg of CT) of NPs compared to saline (n=5-10 per group). (b) Representative heat maps show time spent in different locations of the NORT testing chamber. In the BMT, the latency to locate and enter the escape hole in the acquisition (c) and reversal learning (d) phases was lower in Tat+ mice treated with BDNF-CT vs. saline. Representative diagrams show paths from the starting position (blue dot) to the escape hole (red dot) on day 3 of acquisition phase (e) and trials 2 and 4 of reversal learning phase (f) (top and bottom respectively). The number of reference errors (g, h) was significantly lower in BDNF-CT compared to saline treated Tat+ mice only in the reversal learning phase (h) of BMT. Data are represented as the mean. Error bars represent S.E.M. Overall treatment differences in the BMT are indicated by # p<0.05 and ## p<0.01. Between- subjects post hoc analysis in the NORT is indicated by *p<0.05.

FIGS. 8A-8L. Improvements in memory and cognitive flexibility during BMT reversal leaning task are associated with enhanced hippocampal synaptogenesis and dendritic integrity. The mean latency percent change from the maximum time allowed to find an escape hole in the BMT reversal learning phase (Reversal Latency % Change) versus the mean CA1, DG and CA3 hippocampal SYP (a-c) and MAP2 (d-f) immunoreactivities (IR, normalized to Tat+/Sal controls) are shown. The reference errors least square means versus the mean CA1, DG and CA3 hippocampal SYP (g-i) and MAP2 (j-1) immunoreactivities (normalized to Tat+/Sal controls) are shown. Correlation Coefficients (r) were determined by calculating Pearson product-moment correlations for CA1, DG and CA3 hippocampal regions. Significant correlations (P<0.05) are indicated by*. FIGS. 9A-9D. BDNF-CT significantly increased TrkB expression and signaling.

Tat+ mice received Dox (100 mg/kg/d, i.p.) with either i.n. Sal (Tat+/Sal) or BDNF-CT (Tat+/BDNF-CT, 0.3 mg/kg of BDNF & 2.4 mg/kg of CT) for 4 days. Tat− controls received i.p. and i.n. saline (Tat−/Sal). Western blot analysis of hippocampal tissues showed the increased expression of the full-length TrkB (TrkB) (a) and pTrkB (b) and the increased pTrkB/Trk ratio (c) in BDNF-CT vs. Sal treated Tat+ mice (***p<0.001), as well as Tat− mice (### p<0.001). Representative images of WB bands (d) are shown. Data are shown as the mean % change from Tat−/Sal controls represented by a dotted line (n=4 per group). Error bars represent S.E.M.

FIGS. 10A-10B. Tat expression in the hippocampus of Dox-treated iTat mice was not significantly altered by BDNF-CT. Tat+ mice received Dox (100 mg/kg/d, i.p.) with either i.n. Sal (Tat+/Sal) or BDNF-CT (Tat+/BDNF-CT) for 4 days. Tat− controls received i.p. and i.n. saline. (a) Western blot analysis of hippocampal tissues showed the upregulaton of Tat in Tat+ (Tat+/Sal and Tat+/BDNF-CT) mice vs. Tat−/Sal mice. BDNF-CT treatment had no effect on Tat protein levels in Tat+ mice. Data are shown as the mean % change from Tat−/Sal controls represented by a dotted line (n=4 per group, ### p<0.001). Error bars represent S.E.M. (b) Representative WB images of Tat 22 kDa bands used for this analysis are shown.

FIGS. 11A-11D. BDNF-CT effects on mouse speed and path efficiency in the BMT. Average speed and path efficiency during the acquisition (a,b) and reversal learning phases (c,d) of the Barnes maze tests are shown. In the acquisition phase, average speed was higher in Tat+ mice that received BDNF-CT vs. saline (Sal) (a). BDNF-CT treatment did not have a significant effect on path efficiency (b). In the reversal learning phase, no overall BDNF-CT effect on average speed was found (c). BDNF-CT-treated Tat+ mice had higher path efficiency (d) compared to saline treated Tat+ mice. Significant treatment effects are indicated by # p<0.05. Error bars are S.E.M.

FIGS. 12A-12B. BDNF-CT did not impair motor function. C57BL/6J mice received i.n. saline (n=10) or BDNF-CT (n=10) daily for 7 days. Open Field (OF) testing was performed on the 7th day and lasted 20 min. The average speed (a) and distance traveled (b) were not significantly different in BDNF-CT vs. saline treated healthy C57BL/6J mice. Error bars represent S.E.M.

FIGS. 13A-13B. BDNF-CT specificity. Rhodamine labeled clathrin nanoparticles with intact BDNF (a) but not with BDNF blocked with TrkB-IgG chimera (b) bind in vitro to the mouse TrkB in hippocampal slices. Scale bar is 50 μm.

FIGS. 14A-14D. Clathrin nanoparticle (CNP). (a) A diagram of a clathrin triskelion (CT) with BDNF and anti-DAT antibody (DATab) conjugated to its heavy chains (CHCs) via PEGs. (b) ELISA confirmed 1 molecule of DATab and 3 molecules of BDNF were attached to CT. Dynamic light scattering (DLS) showed the mean hydrodynamic radius (RH) of unconjugated CT to be 16.8±5.6 nm (top). The mean RH increased to 42.13±14.8 nm after BDNF and DATab conjugation to CT via PEGs (bottom). CNPs with DATab (c) but not with IgG (d) labeled in vitro mouse DAT in striatal slices confirming CNP specificity.

FIGS. 15A-15I. CNP brain distribution. Fluorescent NSH-fluorescein (green) labeled CNPs with DATab were clearly detected in the mouse striatum (STR) (a) and substantia nigra (SN) (b) but not in the frontal cortex (FC) (c) 4 h after i.n. delivery. Brains were also collected from iTat mice at 4 and 6 h after i.n. delivery of radioactive [3]H-CNP with DATab/BDNF. CNP concentrations in the STR (d), SN (e) and FC were assessed. Data are represented as the mean % of the injected dose per gram (% ID/g) of tissue (n=3/group). Error bars show S.D. Striatal sections with DAT-antibody were visualized using Rhodamine-labeled secondary antibody. Fluorescent microscopy shows DAT (red) (g) and intranasally delivered CNPs (green) (h) co-localized with DAT (yellow) (i). The scale bar is 25 μm.

FIGS. 16A-16D. CNPs rescue TH+ fibers from Tat-induced toxicity in the striatum. Tat+ male mice received i.p. Dox and i.n. treatments (Sal or CNP) daily for 7 days. A global control group (Tat−) received only saline. All mice were sacrificed on the 8th day. Striatal sections were evaluated for tyrosine hydroxylase (TH)+ staining (red, a, b, c). TH densities (d) are expressed as the % of saline treated Tat− controls and the mean percentage was higher in CNP-treated vs. saline treated Tat+ mice. Data are represented as the mean percentage. Error bars represent S.E.M. (n=4 per group; *<0.05).

FIGS. 17A-17C. CNPs rescue motor behavior from Tat-induced toxicity. (a) Tat+ male mice (n=8-11/group) received i.p. Dox (100 mg/kg/d) at 9 am for 7 days. Tat+ mice also received i.n. treatments (CNP or Sal) daily. Rotarod and forelimb grip tests were performed on the 7th day. All mice were sacrificed on the 8th day. CNP-treated Tat+ mice exhibited improved rotarod performance (b) and higher grip strength (c) compared to Sal treated Tat− mice. Significant effects are shown (*p<0.05). Error bars are S.E.M.

### DETAILED DESCRIPTION

Provided herein are compositions and methods for treating neurodegenerative disorders (e.g., Alzheimer's Disease, HIV-Associated Neurocognitive Disorder (HAND), Parkinson's, and Huntington's diseases), neuroinflammatory diseases (e.g., amyotrophic lateral sclerosis and multiple sclerosis), stroke, traumatic brain injury, and psychiatric disorders (e.g., depression). In one aspect, provided herein are compositions comprising neurotrophic factor (referred to herein as a NTF) linked to a clathrin nanoparticle. In another aspect, provided herein are methods for treating a human subject having or at risk for developing a neurodegenerative disorder, neuroinflammatory disease, stroke, traumatic brain injury, or psychiatric disorder comprising administering to the human subject a composition comprising a NTF to a clathrin nanoparticle.

Examples shown herein demonstrate that clathrin-triskelia (CT) nanoparticles efficiently transported BDNF to hippocampus after i.n. administration and that BDNF concentrations were sufficient to induce beneficial molecular, cellular and behavioral effects in mice. BDNF-CT targeted TrkB receptors, increased newborn cell survival/proliferation and neurogenesis in the granule cell layer of dentate gyms, and enhanced synaptogenesis and dendritic integrity. BDNF-CT attenuated learning and memory deficits induced by conditional Tat protein expression in Tat+ mice, most likely, as a direct consequence of the molecular effects noted herein.

Intranasal BDNF-CT, but not BDNF or CT alone, increased hippocampal mBDNF levels, indicating that BDNF conjugation to CT is necessary for efficient i.n. delivery of BDNF to the brain. Intranasal BDNF-CT administration also increased proBDNF expression. The proBDNF also may have contributed to the higher mBDNF levels since mBDNF is formed by extracellular plasmin cleavage of proBDNF⁷², and because mBDNF stimulates its own production by increasing proBDNF synthesis and cleavage via a TrkB mediated mechanism⁷³. The mBDNF/proBDNF ratio was higher in BDNF-CT treated Tat+ mice compared to saline-treated Tat+ and Tat− controls. The high mBDNF/proBDNF ratio may be functionally significant since mBDNF exerts pro-survival effects and enhances memory formation and storage²³. ProBDNF binds to pan-neurotrophin receptor p75 (p75^{NTR}) with high affinity and elicits biological effects opposing those of mBDNF⁷⁴. While mBDNF induces long-term potentiation (LTP)⁷⁵⁻⁷⁸ and neurogenesis⁷⁹, proBDNF prompts long-term depression⁸⁰ and neuronal apoptosis⁸¹. Further, conversion of proBDNF into mBDNF regulates memory formation⁸² and is impaired in patients with HIV-associated dementia⁴⁸. Therefore, the mBDNF/proBDNF ratio plays an important role in cell survival and cognitive functioning^{82,83}.

BDNF binding to TrkB receptors elicits receptor dimerization and auto-phosphorylation, which activates intracellular pathways including the phosphatidyl inositol-3 kinase (PI3K)/protein kinase B (Akt) pathway⁸⁴. BDNF not only induces Akt phosphorylation⁸⁵, but also upregulates Akt⁸⁶. The selective increases in pAkt and Akt levels by i.n. BDNF-CT treatment versus other treatments in this study confirms delivery of sufficient concentrations of intact BDNF for PI3K/Akt pathway activation. Tat+ mice treated with BDNF-CT or saline had higher pAkt to Akt ratios compared to saline-treated Tat− mice indicating PI3K/Akt pathway activation. HIV Tat can increase Akt signaling via multiple mechanisms⁸⁷. However, levels of both Akt and pAkt were significantly higher in BDNF-CT vs. Sal treated Tat+ mice, indicating that only BDNF-CT treatment enhanced Akt expression and signaling. By increasing Akt levels, BDNF-CT kept the pAkt/Akt ratio in the normal range, which is important for maintaining normal cell functions and preventing Akt pathway dysregulation⁸⁸.

Our results also show that biological activity of BDNF is preserved even after PEGylation and crosslinking to CT. The results from the confocal microscopy studies indicate that there was abundant and punctate BDNF-CT-Rho staining in DG that co-localized with TrkB staining. Brain distribution of BDNF-CT-Rho corresponds to the previously reported distribution of BDNF in the CNS⁸⁹. These results further confirm delivery of NPs and selective targeting of TrkB receptors. Moreover, BDNF-CT increased full-length TrkB expression and signaling. The full length TrkB receptor was assessed because it contains an intracellular tyrosine kinase domain that rapidly transmits the effects of BDNF binding to a downstream network. The results confirmed that BDNF-CT NP binds to full-length TrkB and triggers its downstream signaling in vivo.

The noninvasive i.n. route was elected for BDNF-CT administration because it circumvents the BBB and provides a direct pathway into the brain parenchyma^{1,2}. The i.n. delivery efficiency of native BDNF to the brain is very low because BDNF is highly charged with an isoelectric point of 10 and is unstable⁹⁰. For example, only 0.0024% of injected BDNF dose per gram of tissue (% ID/g) was found in the rat hippocampus 60 minutes after delivery²⁹. By contrast, the peak hippocampal BDNF concentration achieved 4 h after i.n. BDNF-CT administration in the present study was more than 400-fold higher (1.00% ID/g or equivalent of 100 ng/g), and above the concentration (20 ng/mL) reported to enhance survival of hippocampal neurons in cell cultures^{91,92} and to induce LTP in hippocampal slices⁷⁸. Further, the peak BDNF-CT hippocampal concentration that was detected is several magnitudes higher than previously reported using other BDNF delivery methods. For example, chitosan-enhanced delivery of BDNF across the nasal barrier achieved 0.0096% ID/g in the rat hippocampus⁹³. Of note, after i.n delivery, a BDNF concentration of 0.0057% ID/g (or 4 ng/mL)²⁹ upregulated frontal cortex pAkt in rats. In cell cultures, BDNF concentrations (<5 ng/mL) are shown to induce activation of Akt, but not neuroprotection⁹⁴. By contrast, nonsignificant changes in hippocampal BDNF, Akt and pAkt levels were detected after daily i.n. delivery of native BDNF for 4 days. The apparent discrepancy between studies could be due to differences in animal models and species, BDNF exposure duration, brain region assessed, and/or BDNF dose.

BDNF-CT increased DG GCL newborn cell survival, proliferation, and neurogenesis in Tat+ mice, compared to saline treated Tat+ controls or Tat− mice. This is consistent with the observed significant increases in mBDNF, Akt and pAkt levels in BDNF-CT treated Tat+ mice versus saline treated Tat+ and Tat− mice. Further, these results are consistent with BDNF's role in neurogenesis⁹⁵ and with previous studies reporting increased numbers of BrdU+ cells following BDNF infusion^{30,32,96} and increased numbers of Ki67+ and DCX+ cells following BDNF lentiviral vector infusions³⁶. Importantly, by using noninvasive i.n. BDNF-CT delivery, we were able to achieve results comparable to those achieved in studies of direct hippocampal infusion of BDNF.

Tat+ mice had decreased Ki67+ cell densities compared to Tat− mice. Also, Tat+ mice had impaired hippocampal neurogenesis, as reflected by the decreased percentage of BrdU+ cells that are DCX+ in the GCL. Densities of BrdU+ cells in the GCL were slightly, but not significantly lower in Tat+ vs. Tat− mice, indicating a relatively intact survival of newborn cells after 7 days of Tat induction. The results are consistent with results reported in previous studies⁹⁷⁻¹⁰⁰. Because previous iTat mouse studies⁹⁹ showed that Tat induced toxic effects on the GLC newborn cell proliferation and differentiation into young neurons, Tat versus Dox effects in this study were not compared. Tat-induced toxic effects on neurogenesis were completely reversed by BDNF-CT treatment in this study.

Hippocampal SYP and MAP2 immunoreactivities were higher in BDNF-CT treated versus saline treated Tat+ mice, indicating that BDNF-CT treatment enhanced synaptogenesis and dendritic integrity and that these effects were sustained for at least days. These results are consistent with studies documenting increased SYP^{101,102} and MAP2¹⁰³ expressions following BDNF treatment, and with studies reporting protective effects of BDNF against synaptodendritic injury¹⁰⁴. HIV-1 infection causes synaptodendritic injury, and decreased SYP and MAP2 biomarkers have been associated with higher viral load and impaired cognitive function in HAND¹⁰⁵. Tat exposure decreased SYP^{58,106} and MAP2^{107,108} in cortical and hippocampal neuronal cultures. Also, Tat injection into the rat hippocampus¹⁰⁹ and Tat expression in Dox treated iTat mice⁹⁹ lowered MAP2. Tat decreases SYP and MAP2 levels through mechanisms correlated with BDNF down-regulation⁵⁸. BDNF-CT treatment was able to reverse these neurotoxic effects of Tat. Further studies are required to elucidate long-term effects of BDNF-CT on dendritic and/or spine morphology associated with learning and memory in Tat+ mice.

Tat+ mice exhibit learning and memory deficits⁵⁹. As hypothesized, BDNF-CT treatment enhanced spatial learning and memory and cognitive flexibility in Tat+ mice, which are reflected as improved performance in the BMT acquisition and reversal learning phases, respectively. In the reversal learning phase, BDNF-CT treated mice took less time to find a new escape hole location, used more efficient routes to the escape hole and made fewer reference errors. BDNF-CT treatment increased speed in Tat+ mice only in the acquisition phase, but not in reversal learning phase. This effect was task specific. BDNF-CT did not impair motor function in the OF test and this finding is consistent with previous preclinical BDNF toxicity studies¹¹⁰. Since BDNF-CT treatment was administered only during Tat induction and terminated before behavioral testing, the results indicate that i.n. BDNF-CT treatment exerts durable cognitive-enhancing effects that are sustained for at least 5 days.

Enhanced recognition memory performance also was observed in BDNF-CT treated Tat+ mice, as reflected by a higher NORT % recognition index (RI). The findings of a BDNF-CT dose-effect relationship on NORT may indicate that a threshold BDNF dose is necessary for BDNF to act as a cognitive enhancer in Tat+ mice. This underscores the importance of using methods capable of achieving efficient brain BDNF delivery. The BMT and NORT performance deficits rescued by BDNF-CT are consistent with the reported role of BDNF in learning and memory²¹⁻²⁴. By using noninvasive i.n. BDNF-CT delivery, we were able to achieve results similar to those reported in studies of direct BDNF brain infusion in rodent dementia models^{25,111}.

Without wishing to be bound by theory, the BDNF-CT induced cognitive enhancement is likely due to the molecular changes that were detected in BDNF-CT treated Tat+ mice. These include increased PI3K/Akt pathway signaling, and enhanced SYP and MAP2 expressions. PI3K/Akt pathway activation is directly correlated with spatial memory formation, as antisense BDNF inhibited activation of PI3K/Akt signaling and inhibited spatial learning¹¹². Loss of hippocampal SYP¹¹³ or MAP2¹¹⁴ proteins correlates with spatial memory impairments. Tat decreases SYP and MAP2 levels and impairs neurite outgrowth through mechanisms correlated with BDNF down-regulation⁵⁸. The SYP and MAP2 increases that were observed in the hippocampus of Tat+ mice treated with BDNF-CT correlated with decreased errors and improved BMT reversal learning task performance. Because immature neurons identified by DCX+ staining are not fully incorporated into hippocampal networks, they may not have directly contributed to the positive behavioral outcome after BDNF-CT treatment. However, BDNF-induced neurogenesis, synaptogenesis, and synaptic plasticity is known to have a positive effect on memory²¹⁻²⁴.

The present study represents the first in vivo demonstration of noninvasive clathrin-mediated i.n. delivery of BDNF to the mouse brain resulting in neurorestorative and cognitive-enhancing effects in an animal model of Tat neurotoxicity in HIV/neuroAIDS. These results might also be applicable to other viruses that can induce neuronal damage and cognitive deficits (e.g., COVID-19)¹¹⁷. The convergence of molecular, cellular, and behavioral effects of BDNF-CT administration indicates that clathrin NPs enable efficient delivery of BDNF into the brain. Thus, clathrin nanotechnology may be able to enhance neuronal regeneration and plasticity, and may help restore brain function more efficiently than existing treatments. This nanotechnology has the potential to become a powerful tool in regenerative medicine, and in the future may lead to the development of targeted drug delivery systems, and also neuronal protection and repair platforms.

### Clathrin Nanoparticles

The clathrin nanoparticles described herein can include clathrin cages, clathrin triskelia, or one or more clathrin heavy chains. A clathrin triskelion consists of three clathrin heavy chains (CHC) and three clathrin light chains (CLC) interacting at their C-termini, each heavy chain (about 190 kDa) has a light chain (about 25 kDa) bound to it. The three heavy chains provide the structural backbone of the clathrin cage, and the three light chains are thought to regulate the formation and disassembly of a clathrin cage. The CHC leg can be divided up into three regions: a proximal region that is close to the central vertex and is where the light chain attaches, a distal region, and the N-terminal domain at the end of the leg. An exemplary sequence of the human CHC is shown below (SEQ ID NO: 1):

| | | | | | | |
|---|---|---|---|---|---|---|
| 1 | MAQILPIRFQ | EHLQLQNLGI | NPANIGFSTL | TMESDKFICI | REKVGEQAQV | VIIDMNDPSN |
| 61 | PIRRPISADS | AIMNPASKVI | ALKAGKTLQI | FNIEMKSKMK | AHTMTDDVTF | WKWISLNTVA |
| 121 | LVTDNAVYHW | SMEGESQPVK | MFDRHSSLAG | CQIINYRTDA | KOKWLLLTGI | SAQQNRVVGA |
| 181 | MQLYSVDRKV | SQPIEGHAAS | FAQFKMEGNA | EESTLFCFAV | RGQAGGKLHI | IEVGTPPTGN |
| 241 | QPFPKKAVDV | FFPPEAQNDF | PVAMQISEKH | DVVFLITKYG | YIHLYDLETG | TCIYMNRISG |
| 301 | ETIFVTAPHE | ATAGIIGVNR | KGQVLSVCVE | EENIIPYITN | VLQNPDLALR | MAVRNNLAGA |
| 361 | EELFARKFNA | LFAQGNYSEA | AKVAANAPKG | ILRTPDTIRR | FQSVPAQPGQ | TSPLLQYFGI |
| 421 | LLDOGQLNKY | ESLELCRPVL | QQGRKOLLEK | WLKEDKLECS | EELGDLVKSV | DPTLALSVYL |
| 481 | RANVPNKVIQ | CFAETGQVQK | IVLYAKKVGY | TPDWIFLLRN | VMRISPDQGQ | QFAQMLVQDE |
| 541 | EPLADITQIV | DVFMEYNLIQ | QCTAFLLDAL | KNNRPSEGPL | QTRLLEMNLM | HAPQVADAIL |
| 601 | GNOMFTHYDR | AHIAQLCEKA | GLLQRALEHF | TDLYDIKRAV | VHTHLLNPEW | LVNYFGSLSV |
| 661 | EDSLECLRAM | LSANIRQNLQ | ICVQVASKYH | EQLSTQSLIE | LFESFKSFEG | LFYFLGSIVN |
| 721 | FSQDPDVHFK | YIQAACKTGQ | IKEVERICRE | SNCYDPERVK | NFLKEAKLTD | QLPLIIVCDR |
| 781 | FDFVHDLVLY | LYRNNLQKYI | EIYVQKVNPS | RLPVVIGGLL | DVDCSEDVIK | NLILVVRGQF |
| 841 | STDELVAEVE | KRNRLKLLLP | WLEARIHEGC | EEPATHNALA | KIYIDSNNNP | ERFLRENPYY |
| 901 | DSRVVGKYCE | KRDPHLACVA | YERGQCDLEL | INVCNENSLF | KSLSRYLVRR | KDPELWGSVL |
| 961 | LESNPYRRPL | IDQVVQTALS | ETQDPEEVSV | TVKAFMTADL | PNELIELLEK | IVLDNSVFSE |
| 1021 | HRNLQNLLIL | TAIKADRTRV | MEYINRLDNY | DAPDIANIAI | SNELFEEAFA | IFRKFDVNTS |
| 1081 | AVQVLIEHIG | NLDRAYEFAE | RCNEPAVWSQ | LAKAQLQKGM | VKEAIDSYIK | ADDPSSYMEV |
| 1141 | VQAANTSGNW | EELVKYLQMA | RKKARESYVE | TELIFALAKT | NRLAELEEFI | NGPNNAHIQQ |
| 1201 | VGDRCYDEKM | YDAAKLLYNN | VSNFGRLAST | LVHLGEYQAA | VDGARKANST | RTWKEVCFAC |
| 1261 | VDGKEFRLAQ | MCGLHIVVHA | DELEELINYY | QDRGYFEELI | TMLEAALGLE | RAHMGMFTEL |
| 1321 | AILYSKFKPQ | KMREHLELFW | SRVNIPKVLR | AAEQAHLWAE | LVFLYDKYEE | YDNAIITMMN |
| 1381 | HPTDAWKEGQ | FKDIITKVAN | VELYYRAIQF | YLEFKPLLLN | DLLMVLSPRL | DHTRAVNYFS |
| 1441 | KVKQLPLVKP | YLRSVQNHNN | KSVNESLNNL | FITEEDYQAL | RTSIDAYDNF | DNISLAQRLE |
| 1501 | KHELIEFRRI | AAYLFKGNNR | WKQSVELCKK | DSLYKDAMQY | ASESKDTELA | EELLQWFLQE |
| 1561 | EKRECFGACL | FTCYDLLRPD | VVLETAWRHN | IMDFAMPYFI | QVMKEYLTKV | DKLDASESLR |
| 1621 | KEEEQATETQ | PIVYGQPQLM | LTAGPSVAVP | PQAPFGYGYT | APPYGQPQPG | FGYSM |

Residues 1-479 can also be referred to as the N-terminal globular domain region, where residues 24-330 include seven repeat regions, residues 331-394 are a flexible linker region, and residues 449-465 are the binding site for the uncoating ATPase.

Residues 457-507 are a region involved in spindle location and interaction with TACC3. Residues 524-1675 can be referred to as the heavy chain arm region, with residues 524-634 being the distal segment region, and residues 639-1675 being the proximal segment region. Within the heavy chain arm region, residues 1213-1522 is a region involved in binding clathrin light chain, and residues 1550-1675 is the trimerization domain region (See, e.g., Wakeham et al. The EMBO Journal 22(19): 4980-4990, 2003). Additional domain characterizations can be found at least at Greene et al. Traffic 1:69-75, 2000.

Humans have two clathrin light chains (CLCa and CLCb), and both can associate with CHC. An exemplary sequence of human CLCa is shown below (SEQ ID NO: 2):

| | | | | | | |
|---|---|---|---|---|---|---|
| 1 | MAELDPFGAP | AGAPGGPALG | NGVAGAGEED | PAAAFLAQQE | SEIAGIENDE | AFAILDGGAP |
| 61 | GPQPHGEPPG | GPDAVDGVMN | GEYYQESNGP | TDSYAAISQV | DRLQSEPESI | RKWREEQMER |
| 121 | LEALDANSRK | QEAEWKEKAI | KELEEWYARQ | DEQLQKTKAN | NRVADEAFYK | QPFADVIGYV |
| 181 | TNINHPCYSL | EQAAEEAFVN | DIDESSPGTE | WERVARLCDF | NPKSSKQAKD | VSRMRSVLIS |
| 241 | LKQAPLVH | | | | | |

An exemplary sequence of human CLCb is shown below (SEQ ID NO: 3):

| | | | | | | |
|---|---|---|---|---|---|---|
| 1 | MADDFGFFSS | SESGAPEAAE | EDPAAAFLAQ | QESEIAGIEN | DEGFGAPAGS | HAAPAQPGPT |
| 61 | SGAGSEDMGT | TVNGDVFQEA | NGPADGYAAI | AQADRLTQEP | ESIRKWREEQ | RKRLQELDAA |
| 121 | SKVTEQEWRE | KAKKDLEEWN | QROSEQVEKN | KINNRIADKA | FYQQPDADII | GYVASEEAFV |
| 181 | KESKEETPGT | EWEKVAQLCD | FNPKSSKQCK | DVSRLRSVLM | SLKQTPLSR | |

The clathrin triskelia can be purified natural triskelia or recombinant triskelia. Methods of purifying triskelia or generating recombinant triskelia are known in the art. Exemplary methods of purifying triskelia can be found at Keen et al. Cell 16:303-312, 1979 and Kirchhausen and Harrison, Cell 23(3):755-761, 1981. Triskelia of the present disclosure can be those purified, e.g., from bovine brain. Recombinant triskelia can be generated by co-expressing clathrin heavy and light chains in e.g., insect cells, followed by purification. The recombinant heavy and light chains can form triskelia, which in turn can spontaneously assemble into cages (e.g., if transferred into low-salt conditions). Alternatively, recombinant heavy chain and light chains can be expressed separately, and reconstituted. For example, purified clathrin light chain expressed in E.coli can be added to purified heavy chain expressed in insect cells (See, e.g., Rapoport et al. Molecular Biology of the Cell 19(1):405-413, 2008).

Clathrin cages are geodesic assemblies of clathrin triskelia and can include varying numbers of triskelia with different geometries. For example, a clathrin cage can have 28, 36, or 60 triskelia, and has a tetrahedral symmetry, hexagonal shaped D6 symmetry, or football shaped icosahedral symmetry, respectively (See, e.g., Royle, Cell Mol Life Sci 63(16): 1823-1832, 2006).

Clathrin cages can be generated by self-assembly of purified or recombinant triskelia. Below pH 6.5, purified clathrin triskelia self-assemble in vitro into polyhedral cages without vesicles, but typically only form cages at physiological pH in the presence of stoichiometric quantities of purified AP-1 or AP-2 adaptor molecules or the neuron-specific assembly proteins AP-180 and auxilin. The adaptors can both trigger clathrin self-assembly and co-assemble with clathrin.

Alternatively, fragments of the triskelion can be combined to form cages. For example, the terminal and distal domains of the heavy chain (TDD, residues 1-1074) and hub domain (residues 1074-1675) of the heavy chain occupied by clathrin light chain (Hub/Lcb) can be expressed (e.g., recombinantly), and assembled under conditions similar to those used for self-assembly of isolated clathrin triskelia (See, e.g., Greene et al. Traffic 1:69-75, 2000). The recombinant hub domains are trimeric structures that reproduce the central portion of the three-legged clathrin triskelion, extending from the vertex to the bend in each leg, comprising the binding sites for clathrin light chain subunits. Without light chain subunits, recombinant hub domains can self-assemble reversibly at physiological pH, while hub domains with bound light chains only self-assemble below pH 6.5, similar to purified triskelion, as light chain subunits inhibit hub assembly at physiological pH. The clathrin cages can have a diameter of at least about 10 (e.g., at least about 20, 30, 40, 50, 60, 70, 80, 90, or 100) nanometers.

The compositions provided herein can include clathrin nanoparticles comprising a clathrin cage made up of a plurality of clathrin triskelia, where one or more heavy chains of a triskelion are linked to a NTF as described herein. The clathrin nanoparticles can also comprise a single clathrin triskelion, where one or more heavy chains of the triskelion are linked to a NTF as described herein. A heavy chain of a triskelion can be linked to up to 5 molecules of the neurotrophic factor. In some instances, the present composition comprises on average three molecules of a neurotrophic factor, wherein one molecule of a neurotrophic factor is linked to one CHC of a triskelion.

In another example, the clathrin nanoparticle can comprise one or more isolated or recombinant CHC proteins (e.g., full length proteins or fragments thereof), and the CHC proteins are linked to a NTF. Fragments of the CHC proteins contemplated herein can include one or more suitable portions of the full length protein, such as the hub domain (e.g., the trimerization domain), the N-terminal domain, or the hub domain attached to the N-terminal domain. Recombinant CHC proteins that are at least 70% identical (e.g., at least 75%, 80%, 82%, 84%, 86%, 88%, 90%, 91%, 92%, 93%, 94%, 95%, 96%, 97%, 98%, or 99% identical) to SEQ ID NO: 1 or a portion thereof are contemplated herein.

For example, the composition can consist of (1) one isolated or recombinant CHC protein or portions thereof linked to a NTF; or (2) two or more isolated or recombinant CHC proteins or portions thereof, where the NTF are linked to the same or different CHC proteins. In some instances, one or more of the CHC proteins can each be bound to one CLC.

The average size of the clathrin nanoparticles provided herein can be any size that is suitable for crossing the blood brain barrier. For example, the nanoparticles can have a cross-sectional diameter of between 1-100 nm (e.g., about 10-80, 20-60, or 30-50 nm). The clathrin nanoparticles provided herein can further include a fluorescent label, a radiolabel (e.g., ¹⁴C, ³H, and ¹⁵³Gd), or an ion-oxide label, which can be helpful in detecting the nanoparticles in vivo.

### Neurotrophic Factors

Neurotrophic factors (NTFs) are a family of proteins that can induce the survival, development and function of neurons (e.g., sensory and sympathetic neurons) in both the peripheral and central nervous systems. NTFs can activate one or more of the three members of the tropomyosin-related kinase (Trk) family of receptor tyrosine kinases (TrkA, TrkB, and TrkC). In addition, NTFs activate p75 neurotrophin receptor (p75NTR), a member of the tumor necrosis factor receptor superfamily. Through Trk receptors, NTFs activate Ras, phosphatidyl inositol-3 (PI3)-kinase, phospholipase C-gammal and signaling pathways controlled through these proteins, such as the MAP kinases. Activation of p75NTR results in activation of the nuclear factor-kappaB (NF-kappaB) and Jun kinase as well as other signaling pathways. Continued presence of the NTFs is required in the adult nervous system, where they control synaptic function and plasticity, and sustain neuronal survival, morphology and differentiation.

NTFs suitable for linking to the clathrin nanoparticles described herein include NT-5. Others include, but are not limited to: nerve growth factor (NGF), brain-derived neurotrophic factor (BDNF), neurotrophin-3 (NT-3) and neurotrophin-4 (NT-4), NT-6, neurturin (NTN), persephin (PSPN), artemin (ARTN), ciliary neurotrophic factor (CNTF), and leukemia inhibitory factor (LIF). Additional proteins that regulate neuronal survival and/or other aspects of neuronal development are also contemplated herein, such as but are not limited to glial cell-derived neurotrophic factor.

In some instances, the NTF linked to clathrin nanoparticles described herein is BDNF. BDNF proteins can be unstable and do not easily cross the BBB (See. e.g., Gilmore et al. J Neuroimmune Pharmacol 3(2): p. 83-94, 2008). BDNF has a short in vivo half-life (<5 min) and poor pharmacokinetic profile, which makes treatment with BDNF difficult. However, agents such as antidepressants and mood stabilizers that can increase BDNF levels act on different sites and have multiple side effects (See, e.g., Bhaskar et al. Part Fibre Toxicol. 7: p. 3, 2010). Without wishing to be bound by theory, linking BDNF to clathrin nanoparticles as described herein allows BDNF to be delivered across the BBB.

### Central Nervous System Stimulants

In some embodiments, the clathrin nanoparticle linked to the NTF is additionally linked to other molecules, such as central nervous system (CNS) stimulants. CNS stimulants (CNS stands for central nervous system) are medicines that stimulate the brain, speeding up both mental and physical processes. They increase energy, improve attention and alertness, and elevate blood pressure, heart rate and respiratory rate. They decrease the need for sleep, reduce appetite, improve confidence and concentration, and lessen inhibitions. Exemplary CNS stimulants include methylphenidate, dextroamphetamine, pemoline, phentermine, phendimetrazine.

### MRI Imaging Agent

In some instances, an MRI imaging agent is added to a clathrin-BDNF-composition, which can also be linked to a CNS stimulant to make it a theranostic. In some instances, BDNF is optionally not included and an MRI imaging agent is added to a clathrin-CNS stimulant composition thereby making a targeted MRI diagnostic. In both instances, a contrast agent can be included, which can include chelates containing paramagnetic metal ions such as Gd (III), Mn (II). Gadolinium contrast agents can be divided according to whether A) the carrier ligand is linear or macrocyclic and B) whether they are ionic or non-ionic, leading to four groupings:
linear—ionic
   Gd-DTPA, gadopentetate dimeglumine (Magnevist®)
   Gd-BOPTA, gadobenate dimeglumine (MultiHance®)
   Gd-EOB-DTPA, gadoxetic acid disodium salt (Eovist® Primovist®)
   MS325, gadofosveset trisodium (Vasovist® Ablavar®)
linear—non-ionic
   Gd-DTPA-BMA, gadodiamide (Omniscan®)
   Gd-DTPA-BMEA, gadoversetamide (OptiMARK®)
macrocyclic—ionic
   Gd-DOTA, gadoterate meglumine (Dotarem® Artirem®)
macrocyclic—non-ionic
   Gd-HP-DO3A, gadoteridol (ProHance®)
   Gd-BT-DO3A, gadobutrol (Gadovist® Gadavist®)

Alternatively, a contrast agent can include Fe (III) or a superparamagnetic iron oxide (SPIO), monocrystalline iron oxide nanoparticle (MION), cross-linked iron oxides (CLIO), or a magneto-dendrimer.

### CNS Targeting Agent

In some instances, a CNS targeting agent is added to a clathrin composition to facilitate delivery of a NTF, therapeutic agent, and/or MRI imaging agent to a specific region or cell receptor type in the brain. The CNS targeting agent can be an antibody or a ligand. Exemplary CNS targeting antibody agents include: anti-CD11b antibody (complement receptor 3 on activated microglia, activated B and T cells), an anti-TrkB antibody (TrkB receptor), an anti-DAT antibody, an anti-GABA antibody, an anti-SYP antibody, an anti-serotonin transporter antibody, an anti-dopamine-1 antibody, an anti-dopamine-2 antibody, or an anti-dopamine-3 antibody (striatum, frontostriatal, prefrontal). The antibody can be a monoclonal antibody. Exemplary CNS targeting ligand agents include: a DAT ligand, a TSPO ligand, a GABA ligand, a serotonin transporter ligand, a D1 ligand, a D2 ligand, or a D3 ligand.

Activation of CNS microglia (MG) is a key event in response to early pathological changes in HIV-Associated Neurocognitive Disorder (HAND). Synapse loss in hippocampus is an early hallmark of the disease and imaging activated MG responsible for this loss could be crucial for early diagnosis. Increased activation of complement proteins was detected in HAND brain tissues in both preclinical and clinical stages of the disease. Therefore, in some instances, the CNS targeting agent is an antibody that targets activated microglia in the CNS. For instance, the antibody is one of the following: anti-CD11b antibody, anti-TREM2 antibody, IL-6R-antibody, or anti-TNF-α-antibody.

### Linking Methods

The clathrin nanoparticles can be linked, e.g., covalently linked, to the NTFs described herein in a number of ways, such as but not limited to, conjugation, cross-linking, covalent bonding, and biotin-avidin interaction. In some embodiments, the clathrin nanoparticles are linked to the NTFs via functional groups. In some embodiments, the functional groups are part of a polymer. The polymer can be a synthetic polymer, such as, but not limited to, polyethylene glycol or silane, natural polymers, or derivatives of either synthetic or natural polymers or a combination thereof. Hydrophilic polymers are useful for as a linking polymer. The polymer can include polysaccharides and derivatives, including dextran, pullanan, carboxydextran, carboxmethyl dextran, and/or reduced carboxymethyl dextran. The polymers can be functionalized (e.g., including amino, carboxyl, or other reactive groups) for attaching the NTFs or any additional desired moieties to the clathrin nanoparticles. In some instances, the cysteines of a clathrin triskelion can be used to attach PEGs, which are then used to crosslink the NTF. Exemplary methods of pegylating nanoparticles can be found at e.g., U.S. Pat. Nos. 7,291,598; 5,145,684; 6,270,806; and 7,348,030.

Avidin or streptavidin can be attached to the clathrin nanoparticles for use with a biotinylated binding moiety, such as an oligonucleotide or polypeptide. See e.g., Shen et al., Bioconjug. Chem., 1996, 7(3):311-6. Similarly, biotin can be attached to the nanoparticles for use with an avidin-labeled binding moiety. Low molecular weight compounds can be separated from the nanoparticles by ultra-filtration, dialysis, magnetic separation, or other means. The NTFs can be separated from the ligand-nanoparticle conjugates, e.g., by size exclusion chromatography. One, two, or all three of the heavy chains of a triskelion can be linked to a NTF. The NTF can be linked to the same or different heavy chains of a triskelion.

### Methods of Treatment

The present disclosure provides methods for treating neurodegenerative disorders (e.g., Alzheimer's Disease, HIV-Associated Neurocognitive Disorder (HAND), Parkinson's, and Huntington's diseases), neuroinflammatory diseases (e.g., amyotrophic lateral sclerosis and multiple sclerosis), stroke, traumatic brain injury, and psychiatric disorders (e.g., depression) in a subject. Generally, the methods include administering a therapeutically effective amount of the composition described herein, to the subject who is in need of, or who has been determined to be in need of, such treatment. The composition can comprise a clathrin nanoparticle linked to a NTF.

### Subject Selection

The term “subject” as used herein refers to any animal. In some instances, the subject is a mammal. The subject can be a human (e.g., an adult, an adolescent, or a child). The methods can be used in any subject who has or is at risk for developing neurodegenerative disorders (e.g., Alzheimer's disease, HIV-Associated Neurocognitive Disorder (HAND), Parkinson's, and Huntington's diseases), neuroinflammatory diseases (e.g., amyotrophic lateral sclerosis and multiple sclerosis), stroke, traumatic brain injury, and psychiatric disorders (e.g., depression). In some instances, HAND includes HIV-associated dementia (HAD). In some instances, HAND includes asymptomatic neurocognitive impairment. In some instances, HAND includes mild neurocognitive disorder. The methods can include selection of a human subject who has or had a disorder or disease described herein. Methods for identifying or diagnosing such subjects are known in the art. Subject selection can include obtaining a sample from a subject and testing the sample for an indication that the subject is suitable for selection. The subject can be confirmed or identified, for example, by a health care professional, as having had or having a disorder. As used herein the term “sample”, when referring to the material to be tested for the presence of a biological marker using the method of the invention, includes inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used can vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The disorders and diseases described herein can be diagnosed based on the levels of dopamine in e.g., bodily fluids such as the cerebralspinal fluid, or in certain areas of the brain, such as the striatum.

### Administration

In some embodiments, the term “treating” and “treatment” refers to administering to a subject an effective amount of a composition, e.g., a composition comprising a BDNF linked to a clathrin nanoparticle, so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment can improve the disease condition, but is not necessarily a complete cure for the disease. In some embodiments, treatment can be “prophylaxic” treatment, where the subject is administered a composition as disclosed herein (e.g., a composition comprising a BDNF linked to a clathrin nanoparticle) to a subject at risk of developing a neurodegenerative disease as disclosed herein, to thereby reduce the risk of developing the disease. In some embodiments, treatment is “effective” if the progression of a disease is reduced or halted. In some instances, blood, urine or other lab tests can be used for monitoring treatment and recovery.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. It can also refer to a sufficient amount of a clathrin nanoparticle described herein to retard, delay or reduce the risk of progression of a disease or condition, symptoms associated with a disease or condition or otherwise result in an improvement in an accepted characteristic of a disease or condition when administered according to a given treatment protocol. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. In some instances, the NTF (e.g., BDNF) can be administered at a dose of between about 0.1 mg/kg to about 10 mg/kg (e.g., between about 0.1 mg/kg to about 6 mg/kg, between about 0.5 mg/kg to about 2 mg/kg) per body weight of the subject. In some instances, the BDNF is administered at about 1 mg/kg.

The terms “administer,” “administering,” or “administration,” as used herein refers to implanting, absorbing, ingesting, injecting, or inhaling, the inventive drug, regardless of form. In some instances, one or more of the compounds disclosed herein can be administered to a subject nasally or intravenously. For example, the methods herein include administration of an effective amount of compound or compound composition to achieve the desired or stated effect. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, composition or combination of this invention can be administered, if necessary. Subsequently, the dosage or frequency of administration, or both, can be reduced, as a function of the symptoms, to a level at which the improved condition is retained. Patients may, however, require intermittent treatment on a long-term basis upon any recurrence of disease symptoms.

The compositions described herein can be used to target specific regions of the brain. In some instances, administration of the compositions result in enhanced striatal fiber density, improved cognitive and/or motor functions, and/or decrease in addiction-associated hyperlocomotion and neurotoxicity. Administration of the compositions can also result in increased levels of NTFs in the striatum.

### Pharmaceutical Formulations

A therapeutically effective amount of the compositions described herein (e.g., a composition comprising a clathrin nanoparticle linked to a NTF) can be incorporated into pharmaceutical compositions suitable for administration to a subject, e.g., a human. Such compositions typically include the composition and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions, e.g., an inhibitor of degradation of the ligand.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. In one aspect, the pharmaceutical compositions can be included as a part of a kit.

Generally the dosage used to administer a pharmaceutical compositions facilitates an intended purpose for prophylaxis and/or treatment without undesirable side effects, such as toxicity, irritation or allergic response. Although individual needs can vary, the determination of optimal ranges for effective amounts of formulations is within the skill of the art. Human doses can readily be extrapolated from animal studies (Katocs et al., Chapter 27 In: “Remington's Pharmaceutical Sciences”, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

### Combination Therapy

The methods of treating neurodegenerative disorders (e.g., Alzheimer's Disease, HAND, Parkinson's, and Huntington's diseases), neuroinflammatory diseases (e.g., amyotrophic lateral sclerosis and multiple sclerosis), stroke, traumatic brain injury, and psychiatric disorders (e.g., depression) as described herein can further include administering one or more additional therapeutic agent. Exemplary additional agents include aripiprazole, baclofen, bupropion, d-AMP and MPH-SR, dextroamphetamine, gabapentin, ibudilast, methylphenidate, mirtazapine, modafinil, NAC, naltrexone, rivastigmine, and topiramate. Nonpharmacological treatments are also contemplated herein as part of a combination treatment, such as transcranial magnetic stimulation and neurofeedback therapy.

### Kits

The present invention also includes kits, e.g., for use in the methods described herein. In some embodiments, the kits comprise one or more doses of a composition described herein. The composition, shape, and type of dosage form for the induction regimen and maintenance regimen can vary depending on a subject's requirements. For example, dosage form can be a parenteral dosage form, an oral dosage form, a delayed or controlled release dosage form, a topical, and a mucosal dosage form, including any combination thereof.

In a particular embodiment, a kit can contain one or more of the following in a package or container: (1) one or more doses of a composition described herein; (2) one or more pharmaceutically acceptable adjuvants or excipients (e.g., a pharmaceutically acceptable salt, solvate, hydrate, stereoisomer, and clathrate); (3) one or more vehicles for administration of the dose; (5) instructions for administration. Embodiments in which two or more, including all, of the components (1)-(5), are found in the same container can also be used.

When a kit is supplied, the different components of the compositions included can be packaged in separate containers and admixed immediately before use. Such packaging of the components separately can permit long-term storage without losing the active components' functions. When more than one bioactive agent is included in a particular kit, the bioactive agents can be (1) packaged separately and admixed separately with appropriate (similar of different, but compatible) adjuvants or excipients immediately before use, (2) packaged together and admixed together immediately before use, or (3) packaged separately and admixed together immediately before use. If the chosen compounds will remain stable after admixing, the compounds can be admixed at a time before use other than immediately before use, including, for example, minutes, hours, days, months, years, and at the time of manufacture.

The compositions included in particular kits of the present invention can be supplied in containers of any sort such that the life of the different components are optimally preserved and are not adsorbed or altered by the materials of the container. Suitable materials for these containers can include, for example, glass, organic polymers (e.g., polycarbonate and polystyrene), ceramic, metal (e.g., aluminum), an alloy, or any other material typically employed to hold similar reagents. Exemplary containers can include, without limitation, test tubes, vials, flasks, bottles, syringes, and the like. As stated above, the kits can also be supplied with instructional materials. These instructions can be printed and/or can be supplied, without limitation, as an electronic-readable medium, such as a floppy disc, a CD-ROM, a DVD, a Zip disc, a video cassette, an audiotape, and a flash memory device. Alternatively, instructions can be published on an interne web site or can be distributed to the user as an electronic mail.

### EXAMPLES The invention is further described in the following examples, which do not limit the scope of the invention described in the claims.

### Background and Materials/Methods for Examples 1 to 9

### Background

In the following examples, it was tested whether a clathrin NP efficiently delivers BDNF into the brain after i.n. administration, and whether BDNF-clathrin triskelia nanoparticles (BDNF-CT NP) exerts beneficial molecular, cellular, and behavioral effects in an iTat mouse model of HIV/neuroAIDS^{45,46}. Down-regulation of BDNF has been linked to HIV-associated neurodegeneration in animals and humans^{47,48}. In HIV patients, decreased BDNF levels in cerebrospinal fluid (CSF) are strongly correlated with increased severity of neurological disease^{49,50}. HIV Transactivator-of-Transcription (Tat) protein is a HIV regulatory protein that promotes viral replication. Tat is released by HIV-infected cells, interacts with uninfected cells (e.g., neurons) and is linked to HIV-associated neurocognitive disorder (HAND)⁵¹ and neurodegeneration⁵². Tat protein has been found in post-mortem brain tissues of HIV patients⁵³ and in the CSF of patients treated with antiretroviral therapy (ART)⁵⁴. In animal models, Tat expression induces inflammation, apoptosis, gliosis⁵⁵, and oxidative stress⁵⁶. Tat expression also reduces gray matter density⁵⁷, and shortens neurite outgrowth by down-regulation of BDNF⁵⁸. Conditional Tat protein expression also induces learning and memory deficits in iTat mice⁵⁹⁻⁶¹. BDNF reverses Tat-induced neurotoxicity in vitro⁶², but BDNF's effects in vivo have not been tested in iTat mice. It was hypothesized that BDNF-CT would target CNS TrkB receptors, activate downstream signaling pathways, increase hippocampal newborn cell survival and proliferation, enhance hippocampal neurogenesis, synaptogenesis and dendritic integrity, and ameliorate learning and memory deficits. The hippocampal brain region was selected for this study because human postmortem studies showed the highest viral concentrations^{63,64} and a maximal degree of microglial activation and neuroinflammation in the temporal lobe of individuals with HIV⁶⁵. Increased plasma viral load and lower CD+4 T-cell counts were consistently associated with smaller hippocampal volumes in HIV-positive adults worldwide, and therefore it is important to protect and restore hippocampal structure and function early in the course of HIV-1 infection⁶⁶.

### Materials and Methods

### BDNF-CT NP Preparation and Characterization

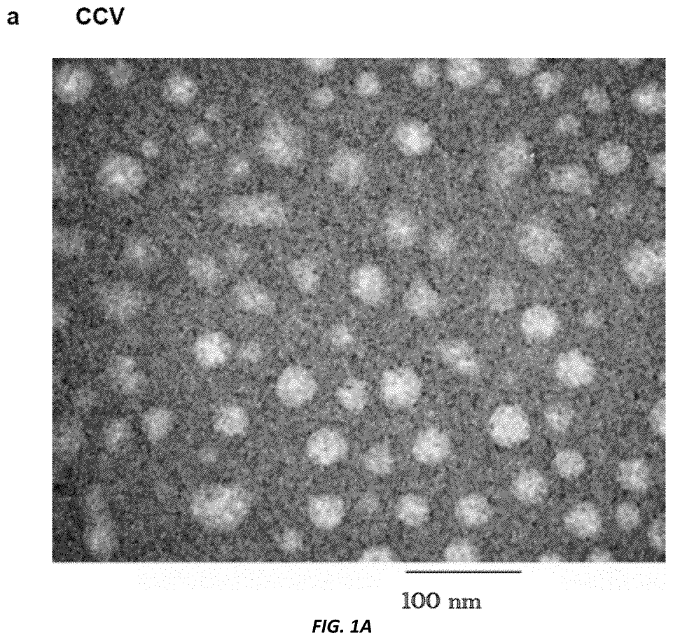

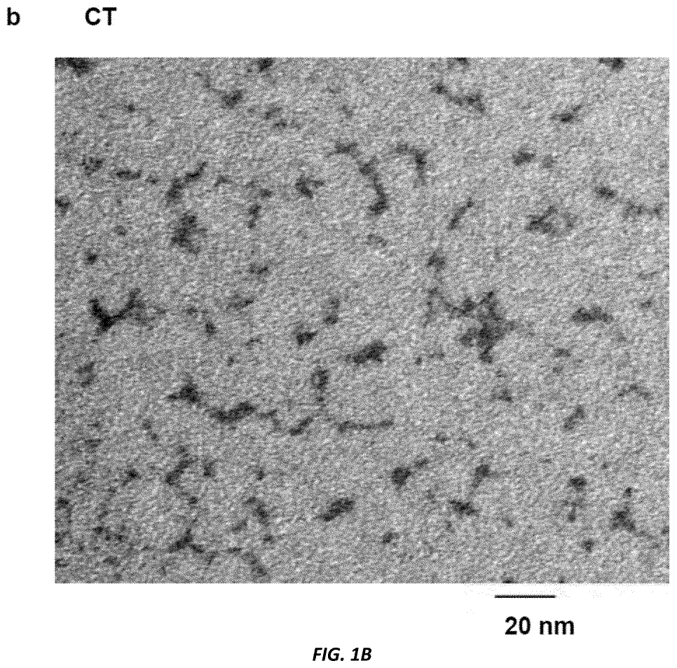

Clathrin-coated vesicles (CCVs) were first isolated from pig brains (Pel-Freez Biologicals, Rogers, AR) and clathrin triskelia (CT) (ExQor Tech., Boston, MA) were then isolated from CCVs using methods described previously^{4,118}CCVs (FIG. 1A) and CT (FIG. 1B) were characterized by Electron Microscopy (1200× Jeol, Tokyo, Japan) using published methods⁴. Concentrations of isolated and purified CT were determined using the Bradford Protein Assay Kit (Bio Rad Laboratories, Hercules, CA, 500-0201) and Synergy HT Multi-Detection Reader (BioTek Instruments, Winooski, VT). Maleimide-Polyethylene glycol-N-Hydroxysuccinimide (Maleimide-PEG-NHS, MW3500, JenKem Tech, Plano, TX) was used to crosslink recombinant human brain-derived neurotrophic factor (BDNF) (Fisher Scientific, Waltham, MA, 50-721-243) to CT at a 3:1 molar ratio, using published methods¹¹⁹. PEGylation of BDNF increases its half-life in the blood and cerebrospinal fluid, and reduces its systemic clearance while minimally affecting biological activity¹²⁰. PEGylated BDNF was reacted with CT in phosphate-buffered saline (PBS, pH 7.4) overnight at 4° C. The maleimide in PEG chains has high affinity for cysteine sulfhydryl groups. The chemical reactivity of CT's cysteines was utilized to attach BDNF-PEGs to CT⁴. Resulting BDNF-CT NPs were purified by ultrafiltration using Nanosep 100K OMEGA devices (Pall Life Sciences, Port Washington, NY, OD100C33) to remove unreacted molecules. BDNF Emax ELISA (Promega, Madison, WI) and Bradford assays were performed to calculate final BDNF and CT concentrations. Sodium dodecyl sulfate polyacrylamide gel electrophoresis (SDS-PAGE) was used to assess NP purity and molecular weight increases following conjugations. Dynamic Light Scattering (DLS) was performed with an LB-550 instrument (Horiba, Kyoto, Japan) to analyze BDNF-CT size and uniformity using published methods⁴.

### Animals and Housing

Adult iTat male 10-14 week-old mice^{45,46} were group-housed (n=2-4/cage) in a controlled environment with a 12/12 h light/dark cycle. iTat mice have a doxycycline (Dox)-inducible Tat transgene under the control of an astrocyte-specific glial fibrillary acidic protein promoter, which produces HIV-Tat protein^{45,46}. Male mice were selected for NP studies because they are more vulnerable to HIV-1 Tat induced CNS damage than females^{45,121}. All mice had ad libitum access to food and water in home cages. Mice were acclimated for a minimum of one-week prior to initiating procedures. All procedures conformed to NIH and National Research Council guidelines on the care and use of laboratory animals.

### Conditional Induction of Tat protein in the Brain

The Tat induction paradigm was based on previous reports⁵⁹. Briefly, to induce Tat expression (resulting in Tat+ mice), iTat mice were treated once daily with an i.p. 100 mg/kg dose of doxycycline hyclate (Dox; Sigma-Aldrich, St. Lois, MO, D9891) for 4 or 7 days⁵⁹ (experimental design is provided in Table 1, see below). Dox solution was prepared fresh each day in sterile 0.9% saline and shielded from light. Hippocampal Tat expression was evaluated using Western blotting.

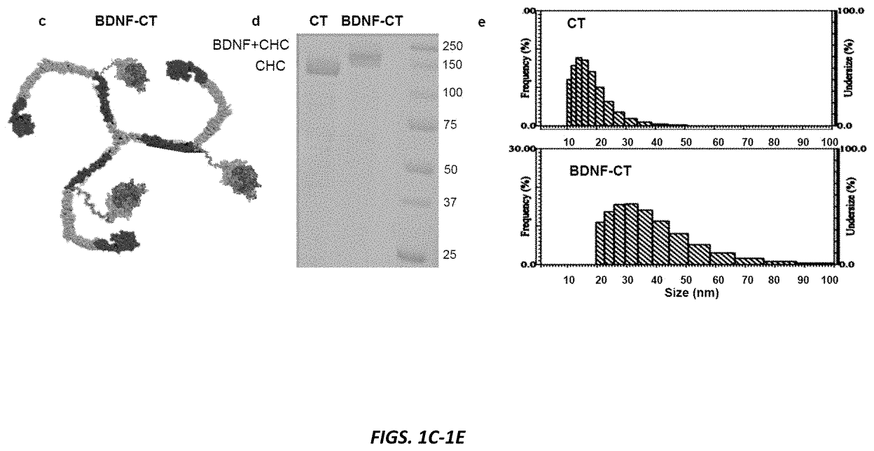

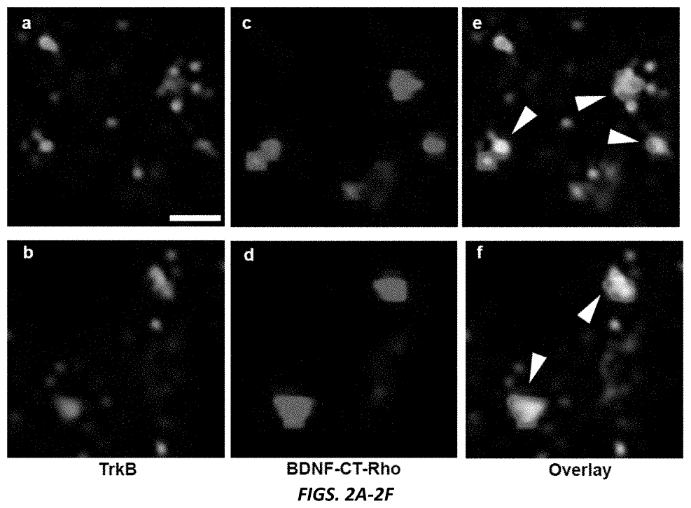

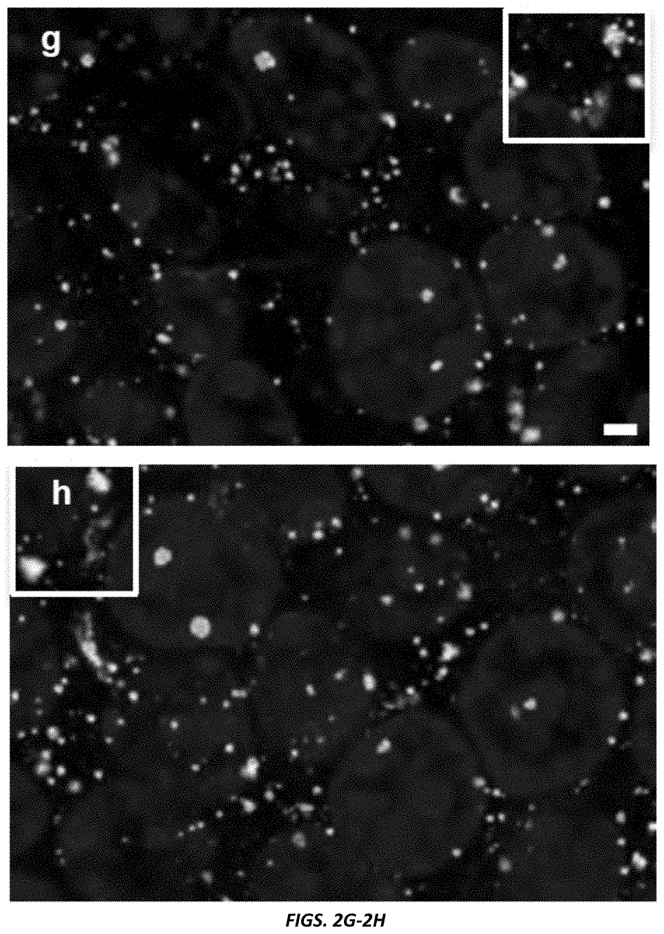

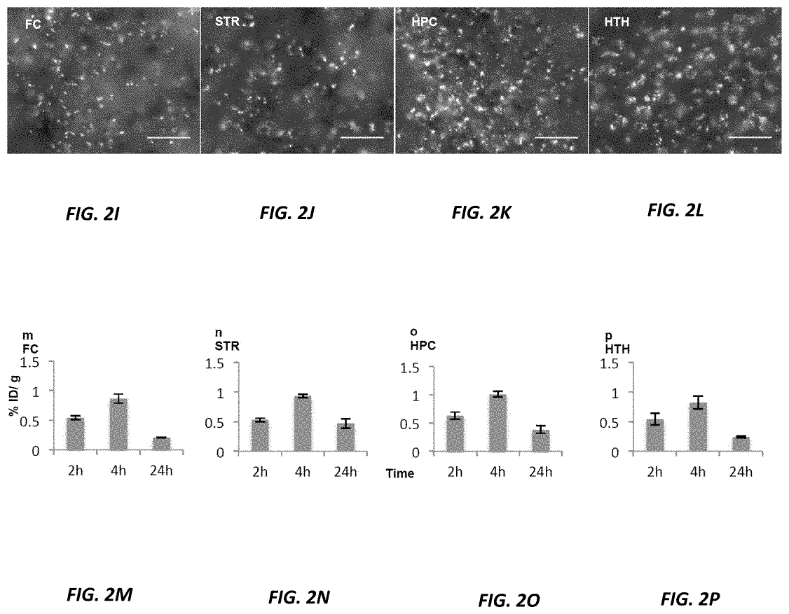

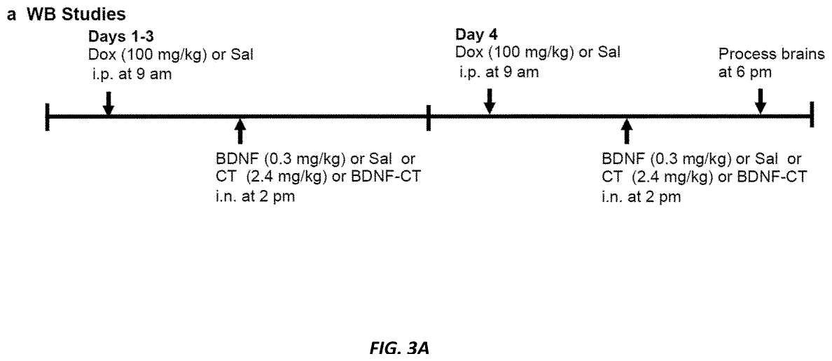

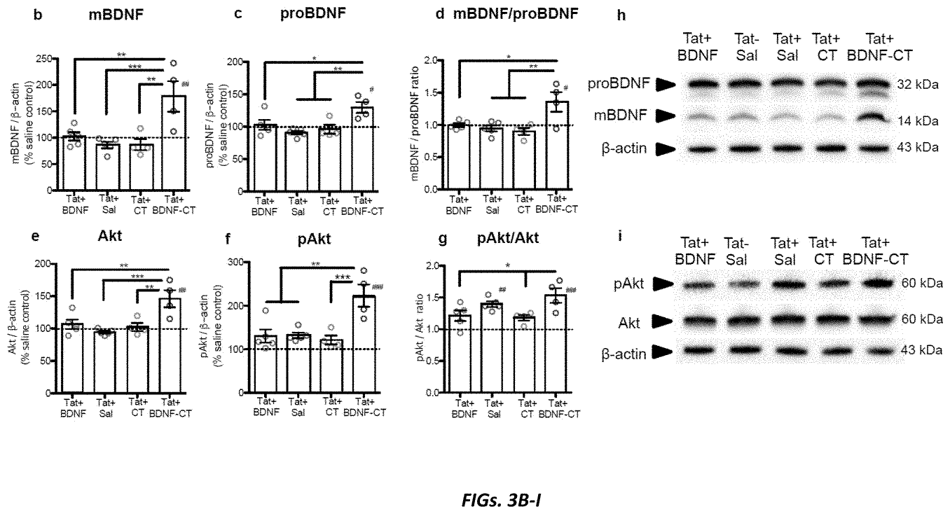

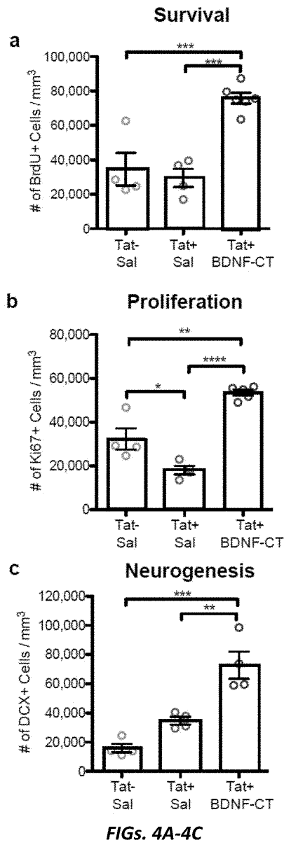

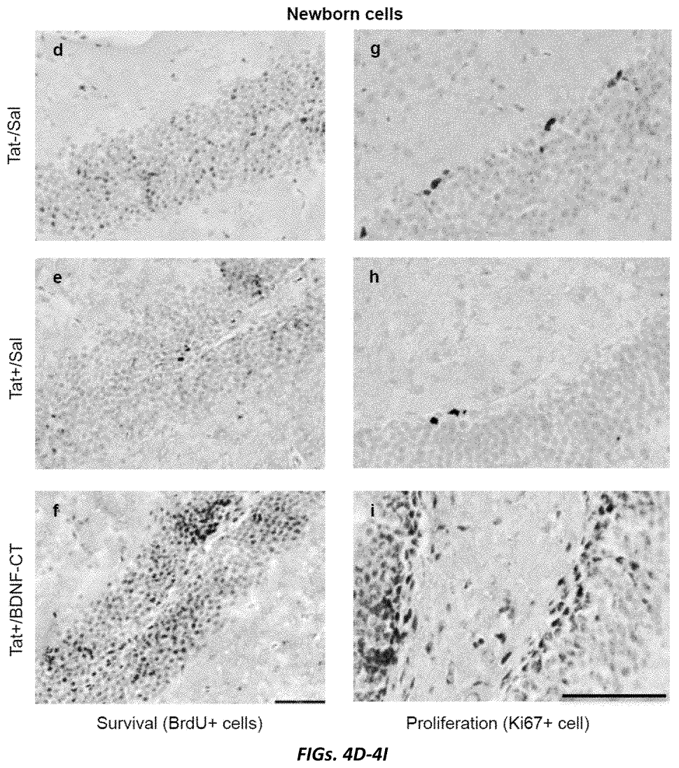

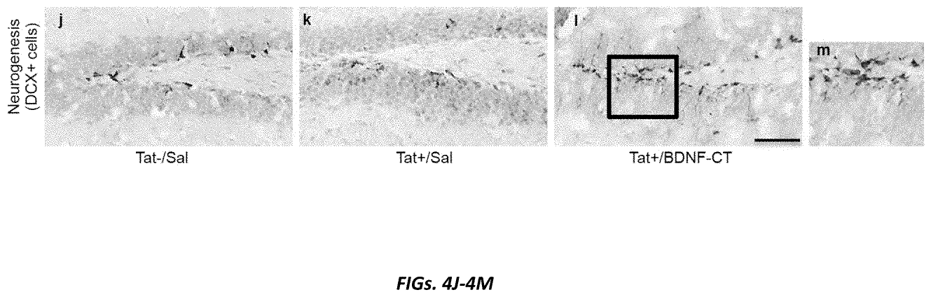

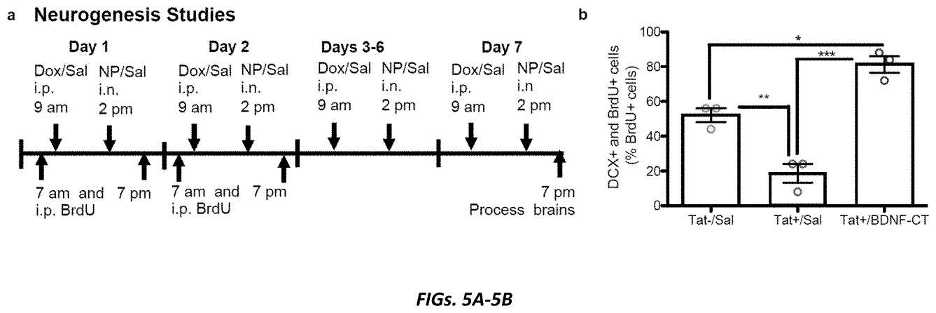

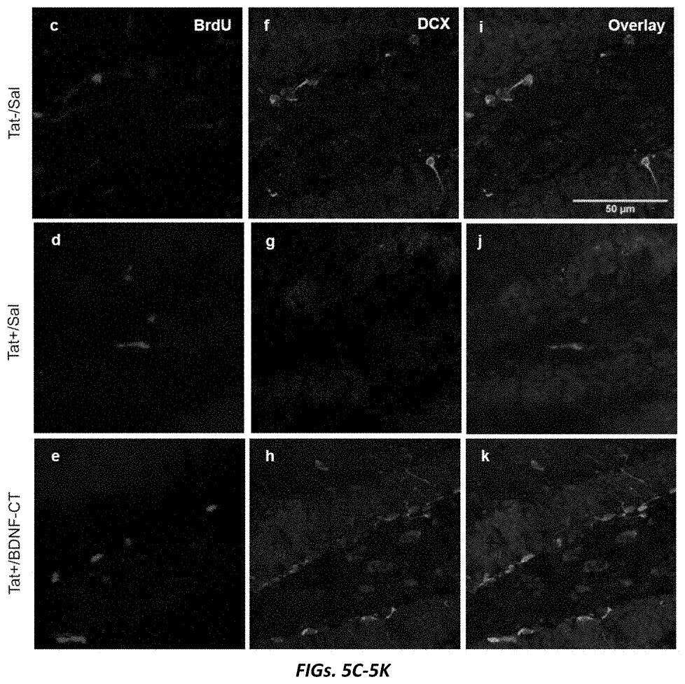

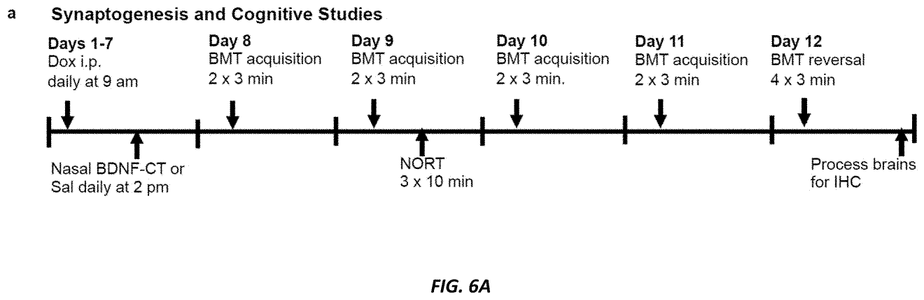

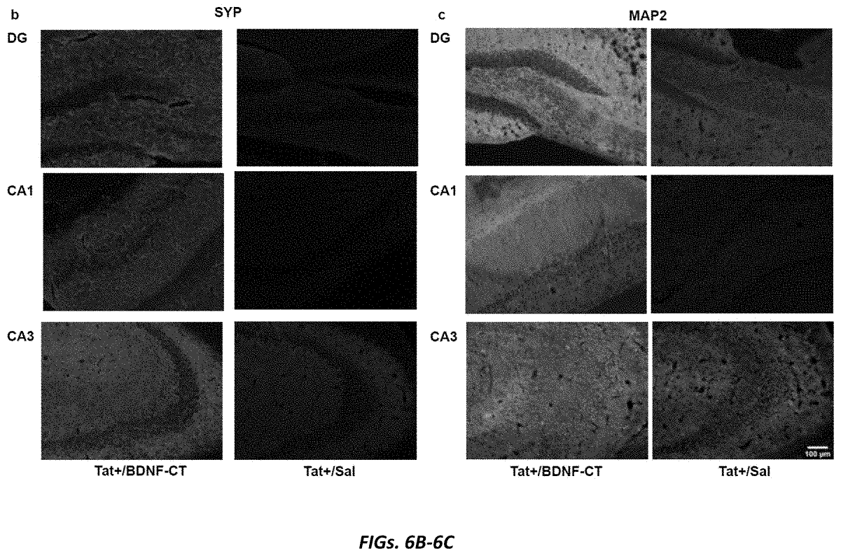

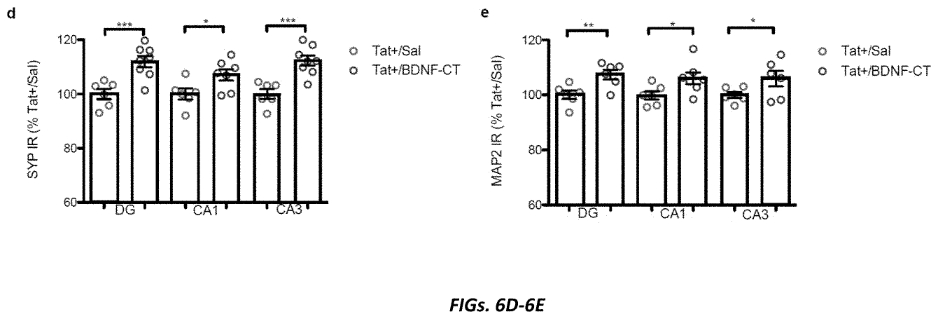

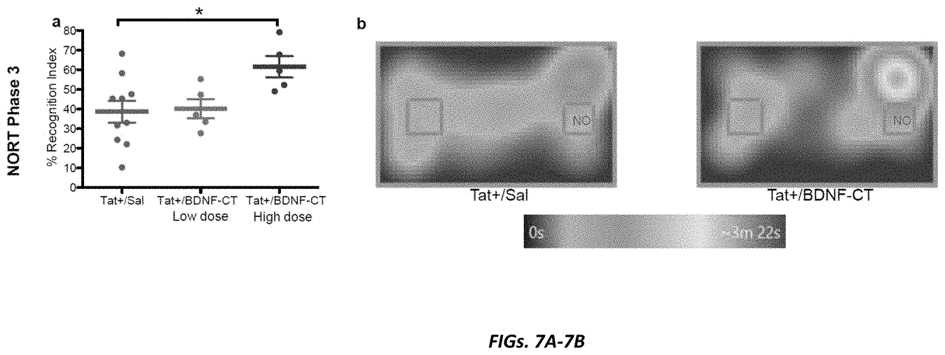

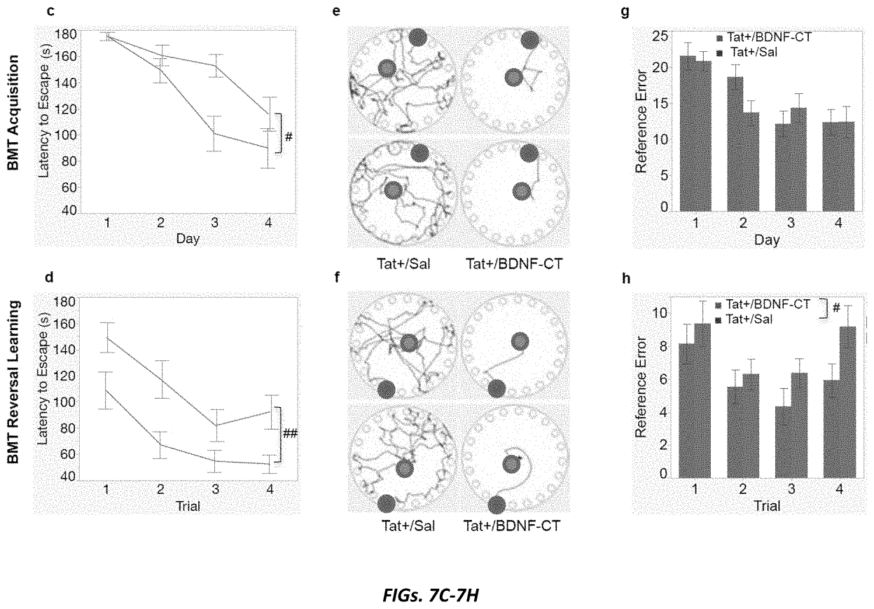

TABLE 1
Summary of Tat induction paradigms and treatment groups.

| | | Intraperitoneal | | |
|---|---|---|---|---|
| | | Saline of | | Tat |
| | Corresponding | Doxycycline | Intranasal | Induction |
| Animal Experiments | FIGS. | (Dox) | Treatments | Period |
| Western blot analysis of | FIG. 3; | Tat−/Sal | Sal | 4 Days |
| mBDNF, proBDNF, pAkt, | Supp. FIGS. | Tat+/Dox | Sal | |
| Akt, pTrkB, TrkB and Tat | 1, 2 & 6 | (100 mg/kg/d) | CT | |
| | | | BDNF | |
| | | | BDNF-CT | |
| BDNF-CT targeting of | FIG. 2 | Tat+/Dox | BDNF-CT-Rho | 7 Days |
| Hippocampal TrkB | Supp. FIG. 5 | (100 mg/kg/d) | or Sal or | |
| | | | ³H-CT-BDNF on day 7 | |
| Analysis of BrdU+, Ki67+, | FIGS. 4 & 5; | Tat−/Sal | Sal | |
| DCX+ cell densities using | Supp. FIG. 7 | Tat+/Dox | Sal | |
| IHC | | (100 mg/kg/d) | BDNF-CT | |
| (BrdU was given during the | | | | |
| first 2 days of Tat-induction) | | | | |
| Analysis of SYP+ and | FIG. 6, 7 & 8; | Tat+/Dox | Sal | |
| MAP2+ IRs using IF | Supp. FIG. 3 | (100 mg/kg/d) | BDNF-CT | |
| Barnes maze and novel | | | | |
| object recognition behavior | | | | |
| testing | | | | |

* Unlike BDNF-CT, unconjugated BDNF did not affect mBDNF, proBDNF, pAkt, or Akt levels or the mBDNF/proBDNF or pAkt/Akt ratios, suggesting that CT are required to deliver effective BDNF concentrations. Therefore, based on a priori go/no-go criteria, BDNF alone groups were not used in subsequent molecular or behavioral experiments.

### Intranasal NP Administration

Mice were sedated using 3% (v/v) vaporized isoflurane, and upon loss of consciousness and righting reflexes, mice were placed in a supine position while horizontally maintaining the ventral side of their heads. A total volume of 40 μl of NP solution or saline was administered i.n. to each mouse by micropipette in 5 μl increments, alternating between each naris every 90 s. Mice took 3-5 minutes to wake up after completing nasal procedures and were immediately returned to their home cage.

### Radioactive-NP Studies

BDNF-CT were radiolabeled with ^{[3]}H-NSP (NET632H005MC, MW=171 Da, PerkinElmer, Shelton, CT) according to a standard labeling method to make ^{[3]}H-CT-BDNF NPs¹²². Briefly, the ^{[3]}H-NSP solution (0.75 mCi) was placed in a glass tube and solvent was evaporated in a gentle stream of nitrogen for 20 min. The CT protein sample (1 mg, MW=650 kDa) in 0.5 mL of borate buffer (pH 8) was transferred to the glass tube, incubated with ^{[3]}H-NSP, and stirred for 4 hours at 4° C. To remove non-reacted particles, protein was dialyzed in PBS buffer (pH 7.4) by using Biotech CE membranes (100 kDa, Spectrum Laboratories, Rancho Dominguez, CA) until the radioactivity in the last 3 buffer solutions was at background level. PEGylated BDNF (100 μg of BDNF) was incubated with ^{[3]}H-CT in PBS buffer (pH 7.4) overnight at 4° C. This method was selected to avoid adding additional positive charges to BDNF, and to preserve its function¹²³. Resulting ^{[3]}H-CT-BDNF NPs were purified by ultrafiltration using Nanosep 100K OMEGA devices (Pall Life Sciences, OD100C33) as described previously. Radioactivity in the ^{[3]}H-CT-BDNF samples was determined using scintillation counting methods, and protein concentration was assessed with Bradford Protein Assay Kit (Bio-Rad Laboratories, 500-0201). iTat male mice received Dox (100 mg/kg/d, i.p.) for 7 days (Tat+ mice). On day 8^{th}, Tat+ mice were given i.n. ^{[3]}H-CT-BDNF. Each mouse received 2.6 μCi of ^{[3]}H-CT-BDNF (10 μg BDNF+30 μg CT). The concentration of ^{[3]}H-CT-BDNF was quantitatively assessed in different brain regions of Tat+ mice at 2, 4, and 24 h after intranasal administration. Brains were rapidly removed and dissected. Brain regions (e.g., olfactory bulb, frontal cortex, striatum, hippocampus, hypothalamus, cerebellum and remaining cortex) were weighed, homogenized, and dissolved in 0.5 mL Solvable (PerkinElmer, 6NE9100) for 4 h at 50° C. Samples then were decolorized by incubation in 0.1 mL of 30% H₂O₂ for 1 h at 50° C. Hionic-Fluor (PerkinElmer, 6013311) scintillation cocktail (4 mL) was added to cooled samples. Concentrations were determined based on radioactivity that was measured by standard scintillation counting methods using a Beckman LC6500 scintillation system.

### Fluorescent-NP Studies

BDNF-CT brain distribution and TrkB receptor targeting were evaluated using double labeling immunofluorescence (IF) with epifluorescence and confocal microscopy. NHS-Rhodamine (Fisher Scientific, 53031) was conjugated to CT using the manufacturer's protocol. Rhodamine-labeled CT were then reacted with PEGylated BDNF (see the NP preparation section) to synthesize Rhodamine-labeled BDNF-CT NPs (BDNF-CT-Rho). Non-reacted particles were removed by ultrafiltration using Nanosep 100K OMEGA devices (Pall Life Sciences, OD100C33) and NP specificity was tested in vitro using mouse hippocampal slices. To block BDNF-CT binding to TrkB in brain slices, a 10-fold molar excess of the recombinant human TrkB-IgG chimera (#688-TK, R & D Systems, Minneapolis, MN) was incubated with NPs (FIG. 13). For in vivo studies, iTat mice received Dox (100 mg/kg/d) for 7 days. On day 8 mice were given a single dose of i.n. BDNF-CT-Rho (0.3 mg/kg of BDNF & 2.4 mg/kg of CT) or saline (n=2-3/group). After 4 h, mice were anesthetized using pentobarbital (120 mg/kg, i.p.), and then transcardially perfused with 4% paraformaldehyde (PFA). Extracted brains were post-fixed in 4% PFA for 24 h and subsequently cryoprotected with 30% sucrose. Brains were embedded in Tissue-Tek O.C.T. compound (Sakura FineTek, Torrance, CA, 4583) and cryosectioned to obtain 20 μm-thick coronal sections. Sections were slide-mounted and stained as described below (see the Immunofluorescent staining).

### NP Delivery into the Brain and NP Signaling

Tat+ mice were randomized into four i.n. treatment groups: saline (Tat+/Sal), BDNF (0.3 mg/kg; Tat+/BDNF), CT (2.4 mg/kg; Tat+/CT), or BDNF-CT (Tat+/BDNF-CT). During a 4-day Tat induction period, animals (Tat+) received Dox (100 mg/kg/d, i.p.) at 9 am. Nasal treatments were administered daily 5 h after Dox i.p. injections. WB studies were performed after 4 days of Dox induction. The 4-day induction paradigm has been shown to significantly increase brain Tat levels in iTat mice⁵⁹. A separate cohort of non-induced iTat mice (Tat−) that had received saline for 4 days (Tat−/Sal) was included as a global control group. Four hours after the last i.n. treatment, mice were anesthetized using pentobarbital (120 mg/kg, i.p.). Their brains were extracted to assess BDNF levels and NP signaling. Using WB analysis, levels of Tat, mature BDNF (mBDNF), proBDNF, TrkB, phosphorylated TrkB (pTrkB) and downstream molecules protein kinase B (Akt) and phosphorylated Akt (pAkt) were determined.

### Western Blot

Hippocampal lysates were prepared¹²⁴ and protein concentrations were determined using the DC™ Protein Assay kit II (Bio Rad Laboratories, 5000112). Proteins were electrophoresed (40 μg/lane) using Mini-Protean TGX Precast Gels (Bio Rad Laboratories, 4569033) and Tris-Glycine SDS buffer (Bio Rad Laboratories, 1610732) at 150 volts for 40 min at 4° C. Resolved proteins were transferred to polyvinylidene difluoride (PVDF) membranes (Bio Rad Laboratories, 1620177) at 16 V overnight at 4° C., and blocked in Tris-buffered saline containing 0.05% (w/v) Tween-20 (TBST) and 5% (w/v) dry milk for 1 h at RT. Membranes then were incubated overnight at 4° C. with the following antibodies: anti-BDNF (1:200, Santa Cruz Biotechnology, Dallas, TX, SC-546), or anti-Akt (1:1000, C6E7, Cell Signaling, Danvers, MA, 4691), or anti-pAkt (1:1000, D9E, Cell Signaling, 4060), or anti-TrkB (1:1000, Santa Cruz Biotechnology, SC-8316), or anti-pTrkB/A (1:1000, Cell Signaling, 9141), or anti-Tat125 (1:500, NT3, 2D1.1 NIH AIDS Reagent Program, 4138). Membranes were washed and incubated with appropriate horseradish peroxidase-conjugated secondary antibodies (1:5000, Cell Signaling) for 1 h, and developed using Clarity Western ECL Substrates (Bio-Rad Laboratories, 170-5060). Membranes were stripped using Restore Plus WB Stripping Buffer (Fisher Scientific, 46428) and re-probed for β-actin (1:1000, C4, Santa Cruz, SC-47778), which was used as a loading control. Because BDNF increases Akt levels86, β-actin was also used as a loading control for pAkt. Images were acquired and analyzed using a Chemi Doc XRS+ molecular imager with Image Lab software (ver. 6.0, Bio-Rad Laboratories). WB band intensities were quantified using the Volume Tools in the Image Lab software and normalized to matched β-actin loading controls. Investigators were blinded to treatment conditions. Percent differences from healthy saline-treated Tat− controls were calculated. (WB images are presented in FIG. 3 and FIGS. 9 and 10). Antibody details are provided in Table 2, see below.)

TABLE 2

| | Target | | | | | | |
|---|---|---|---|---|---|---|---|
| | (anti-) | Dilution | Vendor | Clone | Lot number | RRID | Product ID |
| Primary | TrkB | 1:200 | Santa Cruz | pAb | E0516 | AB_2155274 | SC-8316 |
| (IF) | | | Biotechnology | (H181) | | | |
| | BrdU | 1:100 | BD | mAb | 7324574 | AB_2313824 | 347580 |
| | | | Biosciences | (B44) | | | |
| | DCX | 1:250 | Santa Cruz | pAb | A0714 | AB_2088494 | SC-8066-R |
| | | | Biotechnology | (C18) | | | |
| | SYP | 1:500 | Santa Cruz | mAb | L2016 | AB_628311 | SC-17750 |
| | | | Biotechnology | (D4) | | | |
| | MAP2 | 1:500 | Cell Signaling | pAb | 4 | AB_10693782 | 4542 |
| | | | Technology | | | | |
| Primary | BrdU | 1:100 | BD | mAb | 7324574 | AB_2313824 | 347580 |
| (IHC) | | | Biosciences | (B44) | | | |
| | Ki67 | 1:200 | BD | mAb | 6064956 | AB_393778 | 550609 |
| | | | Biosciences | (B56) | | | |
| | DCX | 1:100 | Santa Cruz | pAb | I0616 | AB_2088494 | SC-8066 |
| | | | Biotechnology | (C18) | | | |
| Primary | BDNF | 1:200 | Santa Cruz | pAb | K1215/G1315 | AB_63094 | SC-546 |
| (WB) | | | Biotechnology | (N20) | | | |
| | Akt | 1:1000 | Cell Signaling | mAb | 17 | AB_915783 | 4691 |
| | | | Technology | (C67E7) | | | |
| | p-Akt | 1:1000 | Cell Signaling | mAb | 19 | AB_2315049 | 4060 |
| | | | Technology | (D9E) | | | |
| | TrkB | 1:1000 | Santa Cruz | pAb | E0516 | AB_2155274 | SC-8316 |
| | | | Biotechnology | (H181) | | | |
| | pTrkB | 1:1000 | Cell Signaling | pAb | 12 | AB_2298805 | 9141 |
| | | | Technology | (4914) | | | |
| | β-actin | 1:1000 | Santa Cruz | mAb | L0117/E0615 | AB_2714189 | SC-47778 |
| | | | Biotechnology | (C4) | | | |
| | Tat | 1:500 | NIH AIDS | mAb | 150254 | AB_1562735 | 4138 |
| | | | Reagent | (NT3.2D1.3) | | | |
| | | | Program | | | | |
| Secondary | Goat-anti- | 1:200 | Fisher | pAb | 1793903 | AB_143165 | A-11008 |
| (IF) | rabbit | | Scientific | | | | |
| | Alexa Fluor | | | | | | |
| | 488 | | | | | | |
| | Goat-anti- | 1:200 | Fisher | pAb | 1797971 | AB_2534072 | A-11004 |
| | mouse | | Scientific | | | | |
| | Alexa Fibor | | | | | | |
| | 564 | | | | | | |
| Secondary | Goat-anti- | 1:200 | Jackson | pAb | 124967 | AB_2338557 | 115-065-003 |
| (IHC) | mouse | | Immuno | | | | |
| | Biotinylated | | Research Lab | | | | |
| | Donkey- | 1:500 | Jackson | pAb | 127296 | AB_2340396 | 705-065-003 |
| | anti-goat | | Immuno | | | | |
| | Biotinylated | | Research Lab | | | | |
| Secondary | Anti-rabbit | 1.5K-10K | Cell Signaling | pAb | 26 | AB_2099233 | 7074S |
| (WB) | IgG, HRP- | | Technology | | | | |
| | linked | | | | | | |
| | Anti-mouse | 1.5K-10K | Cell Signaling | pAb | 31 | AB_330924 | 7076S |
| | IgG, HRP- | | Technology | | | | |
| | linked | | | | | | |

*List of primary and secondary antibodies used. Abbreviations are as follows: Immunofluorescence (IF), immunohistochemistry (IHC), Western blot (WB), monoclonal antibody(mAb), polyclonal antibody (pAb).

### Newborn Cell Survival, Proliferation and Neurogenesis

iTat mice were administered i.p. saline (Tat− controls) or Dox (Tat+) for 7 days to induce Tat expression. Tat+ mice were randomized into two treatment groups and were given either i.n. BDNF (0.3 mg/kg) conjugated to CT (2.4 mg/kg) (Tat+/BDNF-CT), or saline (Tat+/Sal) 5 h after their Dox injections. Tat− mice also received i.n. saline 5 h after i.p. saline injections (Tat−/Sal). BDNF alone and clathrin alone groups were excluded in these experiments, because neither treatment elicited significant molecular changes in the hippocampus in WB experiments. During the first 2 treatment days, all animals also received bromodeoxyuridine (BrdU, 50 mg/kg, i.p.) at 7 am and 7 μm. On day 7, animals were anesthetized at 7 μm using pentobarbital (120 mg/kg, i.p.), and transcardially perfused with 4% PFA to fix brains. Doublecortin (DCX) was evaluated, a marker for neurogenesis that was previously observed in nearly all 7-day-old newborn BrdU-positive (BrdU+) cells in mouse hippocampal GLC¹²⁶. Therefore, a 7-day time point was selected to assess BDNF-CT induced neurogenesis. Brains were post-fixed in 4% PFA for 24 h and then cryoprotected with 30% sucrose. Brains were embedded in Tissue-Tek O.C.T. compound (Sakura FineTek, 4583) and stored at −80° C.

### Immunohistochemistry (IHC) and image analysis

BrdU, Ki67, and DCX were used as indicators of newborn cell survival¹²⁷, proliferation¹²⁸, and neurogenesis¹²⁹, respectively. IHC analysis of the hippocampal GCL was performed to determine 0 densities of BrdU-positive (BrdU+), Ki67-positive (Ki67+) and DCX-positive (DCX+) cells. Slide-mounted coronal sections (20 μm thick) were incubated in 10% methanol (v/v) in PBS with 0.3% hydrogen peroxide for 10 min at room temperature (RT). Sections then were incubated at 99° C. for 15 min in the sodium citrate buffer (10 mM, pH 6) for heat-mediated epitope retrieval (HIER), washed and then blocked in 0.2% Triton X-100/PBS (PBST) with 10% normal serum for 20 min at RT. Sections were probed with well-characterized and widely used primary antibodies: anti-BrdU (1:100, BD Biosciences, 347580), or anti-Ki67 (1:200, BD Biosciences, 550609), or anti-DCX (1:100, Santa Cruz, SC-8066) overnight at 4° C., and then with appropriate biotinylated secondary antibody (1:200-500, Jackson Immuno Research Labs, West Grove, PA) for 30 min at room temperature (RT). The specificity of IHC test was verified by primary antibody omission or inclusion of a non-immune antibody of the same isotype and at the same concentration as the primary antibody. The Vectastain ABC Elite detection system (Vector Labs, Burlingame, CA, PK-7100) was used to visualize staining following manufacturer-recommended procedures. Briefly, sections were incubated for 30 min at RT in avidin—biotin—HRP complex (ABC reagent), and peroxidase activity was detected by incubation with 3,3′ diaminobenzidine (DAB) with nickel for 3 min. Sections were counterstained with hematoxylin (Sigma-Aldrich, MHS16) for 30 s, washed, dehydrated in graded ethanols, cleared and cover-slipped. An investigator blinded to treatment conditions determined the number of positively labeled cells by stereological counting of every 6^{th} section spanning the entire hippocampus, using the Stereo Investigator 11 (MBF Bioscience, Williston, VT) coupled with Zeiss Axio Scope A2 microscope (Zeiss, Thornwood, NY). Settings for gain, aperture, contrast, and brightness were held constant for the analysis of a series of matching sections from the same rostral-caudal level. For each section, GCL boundaries were outlined and the surface area was electronically measured and recorded. Hippocampal GCL surface area was multiplied by tissue thickness to calculate volumes and to determine cell densities.

### Immunofluorescent (IF) Staining

Slide-mounted hippocampal coronal sections underwent HIER treatment for 1 h in citric acid (pH 6) at 99° C. Sections were then cooled to RT, permeabilized, blocked, and incubated with the following primary antibodies: anti-TrkB antibody (1:200, H181, Santa Cruz, SC-8316); anti-BrdU (1:100, B44 BD Biosciences, C:347680); anti-DCX (1:250, C-18, Santa Cruz, SC-8066-R); anti-SYP (1:500, D-4 Santa Cruz, SC-17750) and anti-MAP2 (1:500, Cell Signaling, 4542S) for 24 h at 4° C. Sections were then incubated with appropriate secondary antibodies: goat anti-mouse Alexa Fluor 568 (1:200, Fisher Scientific, A-11004) and/or goat anti-rabbit Alexa Fluor 488 (1:200, Fisher Scientific, A-11008) for 2 h at RT. The specificity of IF test was verified by primary antibody omission or inclusion of a non-immune antibody of the same isotype and at the same concentration as the primary antibody. Slides were then cover-slipped with Vectashield Mounting Medium with DAPI (Vector Labs, H-1200).

### Image Acquisition

Brain sections were imaged with a confocal microscope (Leica Microsystems, Buffalo Grove, IL, TCS SP8). Multiple optical sections spanning 20 μm in the z-dimension were acquired in 1 μm steps at 63× (oil immersion objective). Rhodamine was excited with 561 nm laser and collected at 575 nm emission. Alexa 568 was excited with 578 nm laser and collected at 603 nm emission. Alexa Fluor 488 was excited at 488 nm and collected at 519 nm emission. The Leica Application Suite X (Leica Microsystems) was used to merge images and assess co-labeling. Images were 666 also acquired using 10×/0.3 Ph1, 20×/0.5 Ph2 and 40×/0.75 Ph2 EC Plan-NEOFLUAR M27 667 objectives with ORCA-ER C4742-80 CCD camera (Hamamatsu, Japan) on the Zeiss A1 microscope (Carl Zeiss Inc.) with Micro-Manager software (v1.2, NIH). For LED illumination (100%), 365 nm, 470 nm and 540-580 nm LED modules (Carl Zeiss Inc.) were used for DAPI, Alexa Fluor 488 and Alexa Fluor 568, respectively. Settings for gain, aperture, contrast, and brightness were held constant throughout the study. An investigator blinded to treatment conditions analyzed every 6th hippocampal section from the same rostral-caudal level using an image analysis system (Fiji, v1.52H, NIH). Anatomical regions and landmarks were obtained from the Allen Mouse Brain Atlas (https://atlas.brain-map.org).

### Analysis of BrdU and DCX Markers

Twenty-five BrdU+ cells in the GCL were randomly selected per mouse (n=3/group) based on published protocols¹²⁶, and assessed for co-localization with DCX marker using NIH image analysis system (Fiji, v1.52H, NIH). Briefly, BrdU+ cells were identified in images by processing the red channel in the following manner: background subtraction (Rolling Ball 2000px); Variance (5px) and Mean filters (2px); and a binary image was generated using Renyi's entropy threshold. Finally, particle analysis was used to generate masks that were applied to the raw images. BrdU+ cells in the GLC and within two cell body widths of the GCL in the subgranular zone were automatically counted in every 6^{th} section spanning the entire mouse hippocampus. Twenty-five BrdU+ cells were then randomly selected per animal from this pool of BrdU+ cells using a random number generator. These randomly selected BrdU+ cells were visually inspected, images were pseudo-colored red and green and merged, and cells positive for both the BrdU+ and DCX markers were then counted. The investigator who performed these assessments was blinded to treatment conditions. A confocal microscope (Leica Microsystems, Buffalo Grove, IL, TCS SP8) was used to image and verify double labeled cells. Double-labeled cells were expressed as the percentage of BrdU+ cells. The mean percentages were calculated for treatment groups and compared using ANOVA. (Images were presented in FIG. 5).

### Analysis of SYP and MAP2 Markers

SYP¹³⁰ is a marker of synaptogenesis and MAP2 is a marker of dendritic integrity¹³¹. These markers were evaluated in a cohort of mice (n=6-8/group) that completed cognitive tests by using NIH image analysis system (Fiji, v1.52H, NIH). Every 6^{th} (20 μm thick) hippocampal section was analyzed. Each image represented a randomly selected field of view of 12-bit depth 1344×1024 pixel (x-y plane) of the hippocampal region of interest (ROI) (e.g., DG, CA1 or CA3) that was consistent across animals. Approximately 10 images (10 half-sections) per animal were analyzed for each ROI. The automated Otsu's method was applied to segment the image and find the optimal threshold value of the image by maximizing the weighted between-class variance (e.g., foreground vs. background) and minimizing within-class variance. This method ensures that the area analyzed corresponds to the immunomarker of interest. The automated ImageJ script was then used to analyze segmented images and to measure the mean pixel intensity and area of pixels covered by the immunoreactive staining. The mean intensities were calculated for the hippocampal brain regions (e.g., DG, CA1 and CA3) for each animal and compared between BDNF-CT and saline treated groups using t-tests. (Images were presented in FIG. 6).

### Behavioral Testing of Learning and Memory

During a randomized placebo-controlled preclinical study, animals were videotaped using ANY-maze software (Stoelting Co., Wood Dale, IL), and data were analyzed by investigators blinded to treatment conditions. Effects of NP treatments on hippocampal-dependent learning and memory were examined using the novel object recognition test (NORT) and Barnes maze test (BMT). These tests have been successfully used in iTat mice to show significant differences in learning and memory between Tat+ and Tat− mice⁵⁹ and test procedures were adapted from that study. Tat+ mice were excluded from studies for any one of the following reasons: death, major trauma, anatomical brain and/or other organ malformations, or serious Tat-induced medical problems (e.g., seizures, difficulties breathing, severe gastrointestinal problems, non-healing wounds etc.).

### Barnes Maze Test

iTat mice were treated for 7 days with Dox (100 mg/kg/d, i.p.) and randomized into two groups: a study group (n=14) and a control group (n=16). Four mice were excluded from studies for the reasons listed above. Daily Dox treatments were followed 5 h later by i.n. administration of saline or BDNF-CT NPs (the mean BDNF dose in NPs was 0.23 mg/kg). Following completion of treatments, the BMT (San Diego Instruments, CA) was used to assess spatial learning and memory⁵⁹. Testing was carried out during an acquisition (test days 1-4) and a reversal learning (on test day 5) phase. Before testing began mice were habituated to the BMT apparatus by allowing free exploration for 1 min (without allowing escape), and then by placing them into the unattached escape chamber for 30 s. During the acquisition phase, mice were trained to find and enter the escape chamber during two 3-min acquisition trials, with a 15-min inter-trial interval (ITT). Mice that failed to enter the escape chamber by the end of each trial were gently guided to enter the chamber. Bright light and static radio noise were used to motivate mice to escape the maze. These stimuli were terminated once mice entered the escape chamber. During the reversal learning phase, cognitive flexibility was tested by relocating the escape chamber 150° counterclockwise from its original location and conducting four 3-min trials with 15-min ITIs. All trials were videotaped and analyzed using ANY-maze™ software (Stoelting Co., Wood Dale, IL) to quantify escape latencies, numbers of reference errors, path efficiency and average speed. Path efficiency is calculated by dividing the straight-line distance between the starting and finishing position by the total distance travelled by the animal during a trial. A mouse was determined to have escaped when 100% of his body and all four paws entered the escape chamber. Head deflections into non-escape holes were counted as errors. The maze and the escape chamber were cleaned with 70% ethanol between trials to remove residual secretions and odors.

### Novel Object Recognition Test (NORT)

NORT, a test based on a mouse's natural tendency to explore their environment, was used to test effects of treatments on recognition memory^{59 132}.The NORT consisted of three 10-min phases separated by 10-min ITIs. During each phase, animals freely explored objects placed in rectangular test chambers. For phase 1, two identical objects (e.g., dice) were placed at opposite ends of cages on centerlines, 2 cm away from opposite end walls. During phase 2, the location of one object was moved laterally closer to a side wall. In phase 3, one of the original objects was replaced with a novel object (e.g., a marble), and both objects were placed on the same centerline locations used in phase 1. Phases were videotaped using ANY-maze software (Stoelting Co., Wood Dale, IL) and were analyzed by a blinded observer for time spent on each object, which was defined as physical contact with the object (except via the tail), or sniffing or other manipulation of the object. Data were converted to % recognition index (RI) using the following formula: % RI=(time spent on novel object/time spent on both objects)×100. Objects were thoroughly cleaned with 70% ethanol between phases and subjects to remove secretions and odors. NORT was conducted at the end of the second day of the BMT.

### Evaluation of BDNF-CT Toxic Effects on Motor Functions

To evaluate potential BDNF-CT toxic effects on mouse locomotor activity mice were tested using Open Field (OF) test. C57BL/6J male mice received BDNF-CT (n=10) (n=10) i.n. daily for 7 days and were tested with OF on day 7. Mice were placed in left corner of a square Plexiglas box (40×40×35 cm; Stoelting Co., Wood Dale, allowed to explore it for 20 min. Movement was monitored, recorded and digitally an ANY-maze software. The average speed and total distance traveled were used as locomotor behavior.

### Statistics and Reproducibility

WB and IHC data were analyzed using Prism 7 (GraphPad Software Inc., La Jolla, CA). Behavioral data were analyzed using the JMP Pro 14 software package (SAS Institute Inc., Carey, NC). Data values that were two standard deviations or more away from group means were a priori defined as outliers and excluded from analyses. Significance was defined as P<0.05. One-way analysis of variance (ANOVA) followed by Tukey-Kramerpost hoc testing was used to assess WB and IHC group differences. For SYP and MAP 2 analyses treatment groups were compared using t-tests. For the BMT, overall differences in latency, numbers of reference errors, path efficiency, and average speed were analyzed via a widely used linear mixed-model repeated measure analysis. Akaike information criterion (AIC) was used to assess model fit¹³³ and the model with the lowest AIC value was selected. For BMT the model included the following fixed effects: day (or trial), treatment, and day (or trial)× treatment. RIs from NORT Phase 3 were analyzed for treatment effects using One-Way ANOVA followed by Tukey-Kramerpost hoc tests. Correlations between BMT cognitive performance variables and histological quantifications in the hippocampal regions were performed using Pearson product-moment correlations.

### Example 1 BDNF-CT Characterization

Transmission electron microscopy (TEM) images showed clathrin coated vesicles (CCVs) isolated from pig brains (FIG. 1A), and clathrin triskelia (CT) isolated from CCVs (FIG. 1B). A diagram of three recombinant human BDNF molecules conjugated to CT via Polyethylene glycols (PEGs) is shown in FIG. 1C. The PBS solution of BDNF-CT had 2 mg/mL of CT with 0.25 mg/mL of BDNF and 0.19 mg/mL of PEGs. The total protein concentration was 2.25 mg/mL. Molar ratio of BDNF to CT was 3:1. SDS-PAGE analysis revealed a 29.4 kDa molecular weight increase of the clathrin heavy chain (CHC) that confirmed successful conjugation of BDNF to clathrin triskelion at a 3:1 (BDNF:CT) molar ratio (FIG. 1D). Dynamic Light Scattering (DLS) studies revealed that the mean CT hydrodynamic radius (R_{H}) was 16.8±5.6 nm, consistent with the previously reported value of 16.9 nm⁶⁷ (FIG. 1E, top). A single triskelion has three legs that are flexible, puckered, bent, stretched, close together or extended. Electron microscopy has shown that triskelion legs can vary from 35 to 62 nm in total length after straightening^{68,69}. Atomic force microscopy also confirmed that the legs are flexible along their entire length⁷⁰. Therefore, there is variability in the measurements of triskelion size. BDNF conjugation to CT increased the R_{H} to 35.1±12.6 nm (FIG. 1E, bottom). These results indicate that BDNF-PEGs formed a stable complex with CT, and that free molecules of BDNF-PEGs were not present in the NP solution.

### Example 2. BDNF-CT Targeted TrkB Receptors

Tat+ mice were given a single dose of i.n. BDNF-CT-Rhodamine. Brains were collected after 4 h. Immunostaining with anti-TrkB antibody revealed a robust TrkB immunoreactivity (IR) (green) in the hippocampus (FIG. 2a, b). Fluorescent rhodamine (Rho) signals (red) were observed abundantly throughout the hippocampus in animals that had received BDNF-CT-Rho (FIG. 2c, d). The merged confocal image revealed regions positive for both TrkB and BDNF-CT-Rho throughout the hippocampus. Clear overlapping of the two labels (yellow) was observed at higher magnifications (FIG. 2e, f), and at multiple regions within the dentate gyms (DG) (FIG. 2g, h), indicating that intact BDNF-CT targeted TrkB receptors in vivo.

### Example 3. BDNF-CT Brain Distribution

Qualitative assessment of mouse brains 4 h after i.n. delivery of rhodamine labeled BDNF-CT showed punctate fluorescent deposits in multiple brain regions (FIG. 2i-l). Quantitative assessment of [³H]-BDNF-CT concentrations in brain regions at different time points indicated that peak [³H]-BDNF-CT concentrations were achieved 4 h after i.n. administration (FIG. 2m-p). For example, peak [³H]-BDNF-CT concentrations were found in the frontal cortex (FC) (0.864±0.074% injected dose per gram (% ID/g) of tissue), striatum (STR) (0.932±0.029% ID/g), hippocampus (HPC) (1.008±0.048% ID/g), and hypothalamus (HTH) (0.821±0.108% ID/g).

### Example 4. CT are Required for BDNF Delivery to TrkB Receptors and Downstream Signaling

### 1) Assessments of mBDNF and proBDNF Levels

BDNF-CT significantly increased mature BDNF (mBDNF) levels compared to controls (F_{(4,18)}=8.069, P=0.0007, n=4-5/group; FIG. 3b). Post hoc analysis indicated that hippocampal mBDNF levels were higher in induced iTat (Tat+) mice that had received BDNF-CT compared to saline (P<0.001), CT alone (P<0.01) and unconjugated BDNF (P<0.01). As expected, neither unconjugated BDNF nor CT alone increased mBDNF levels (P>0.05; FIG. 3b). In Tat+ mice, the normalized mean hippocampal mBDNF levels (expressed as percent differences from saline-treated Tat− controls, FIG. 3b) were as follows: 178.2±29% with BDNF-CT; 86.95±10.7% with CT; 86.19±6.11% with saline and 102.6±8.15% with native BDNF treatments. BDNF-CT also increased proBDNF levels F_{(4,18)}=6.239, P=0.0025, n=4-5/group; FIG. 3c). Post hoc analysis revealed increased hippocampal proBDNF levels in Tat+ mice that had received BDNF-CT compared to saline (P<0.01), CT alone (P<0.01) and unconjugated BDNF (P<0.05). Again, proBDNF levels were not altered either by unconjugated BDNF or by CT (P>0.05; FIG. 3c).

BDNF-CT increased the mBDNF to proBDNF ratio F_{(4,18)}=6.554, P=0.002; FIG. 3d). Post hoc analysis revealed an increased mBDNF to proBDNF ratio in Tat+ mice that had received BDNF-CT compared to saline (P<0.01), CT alone (P<0.01) and unconjugated BDNF (P<0.05). Levels of mBDNF (P<0.01), proBDNF (P<0.05), and the mBDNF to proBDNF ratio (P<0.05) were higher in Tat+ mice that had received BDNF-CT compared to saline-treated iTat (Tat−/Sal) control mice.

### 2) Assessment of BDNF-CT Induced Akt Expression and Signaling

BDNF-CT increased hippocampal Akt levels F_{(4,18)}=8.874, P=0.0004, n=4-5/group; FIG. 3e). Akt levels were higher in Tat+ animals that had received BDNF-CT compared to saline (P<0.001), CT alone (P<0.01) and unconjugated BDNF (P<0.01) (FIG. 3e). Similarly, BDNF-CT delivery increased hippocampal phosphorylated protein kinase B (pAkt) levels (F_{(4, 18)}=11.72, P<0.0001, n=4-5/group; FIG. 3f). pAkt levels were higher in Tat+ mice treated with BDNF-CT compared to saline (P<0.01), CT alone (P<0.001) and unconjugated BDNF (P<0.01) (FIG. 3f). Neither CT nor unconjugated BDNF increased Akt or pAkt levels compared to saline (P>0.05). Also, levels of Akt (P<0.01) and pAkt (P<0.001) were higher in Tat+ mice that had received BDNF-CT compared to saline-treated Tat− control mice (FIG. 3 e-f).

BDNF-CT increased the pAkt to Akt ratio F_{(4,18)}=9.314, P=0.0003; FIG. 3g). Post hoc analysis revealed an increased pAkt to Akt ratio in Tat+ mice that had received BDNF-CT compared to CT alone (P<0.05) and unconjugated BDNF (P<0.05). Tat+ mice treated with BDNF-CT (P<0.001) or saline (P<0.01) had higher pAkt to Akt ratios compared to saline-treated Tat− control mice (FIG. 3g) indicating increased activation of Akt signaling pathway. However, levels of both Akt and pAkt were significantly higher in BDNF-CT vs. Sal treated Tat+ mice indicating enhanced Akt expression and signaling only with BDNF-CT treatment. Representative WB images for mBDNF, proBDNF, Akt and pAkt are shown in the FIG. 3h-i.

### 3) Assessment of BDNF-CT Induced TrkB Expression and Signaling

The full-length TrkB protein expression was measured by Western blot (WB) analysis of 145 kDa bands. BDNF-CT increased hippocampal TrkB levels (F_{(2,9)}=47.98, P<0.0001, n=4/group; FIG. 9A). Full-length TrkB levels were higher in Tat+ animals that had received BDNF-CT compared to saline treated Tat+ (P<0.001) and Tat− (P<20 0.001) mice. Similarly, BDNF-CT delivery increased hippocampal phosphorylated TrkB (pTrkB) levels (F_{(2,9)}255.3, P<0.0001, n=4/group; FIG. 9B). Full-length pTrkB levels were higher in Tat+ mice treated with BDNF-CT compared to saline treated Tat+ (P<0.001) and Tat− (P<0.001) mice.

BDNF-CT increased the pTrkB to TrkB ratio F_{(4,18)}=39.78, P<0.0001, n=4/group; FIG. 9C). Post hoc analysis revealed an increased pTrkB to TrkB ratio in Tat+ mice that had received BDNF-CT compared to saline treated Tat+ (P<0.001) and Tat− (P<0.001) mice (FIG. 9C). Representative WB images for pTrkB and TrkB are shown in the FIG. 9D.

### 4) Assessment of Tat Expression

Hippocampal Tat protein expression was measured by WB analysis of 22 kDa bands⁷¹. Daily Dox treatment (100 mg/kg/d, i.p.) over 4 days increased Tat expression in Tat+ mice (F_{(2,9)}=28.20, P=0.0001, n=4/group; FIG. 10A). In the hippocampus, Tat levels were lower in Tat− controls compared to saline-treated Tat+ mice (P<0.001) and BDNF-CT treated Tat+ mice (P<0.001). Hippocampal Tat protein expression was not significantly different between Tat+ mice that had received BDNF-CT compared to saline (P>0.05, FIG. 10A). A faint 22 kDa band was observed in Tat− mice given saline, which may indicate a low level of Tat expression (FIG. 10B).

### Example 6. BDNF-CT Enhanced Newborn Cell Survival, Proliferation and Neurogenesis in the Granule Cell Layer (GCL) of Dentate Gyrus (DG)

### 1) Assessment of Newborn Cell Survival

BDNF-CT treatment enhanced the survival of newborn cells (F_{(2,11)}=21.43, P=0.0002, n=4-6/group, FIG. 4A). In the GCL of DG, bromodeoxyuridine positive (BrdU+) cell densities were higher in Tat+ mice that had received BDNF-CT compared to saline treated Tat+ (P<0.001) and Tat− mice (P<0.001; FIG. 4A).

### 2) Assessment of Newborn Cell Proliferation

BDNF-CT treatment increased the proliferation of newborn cells (F_{(2,10)}=38.54, P<0.0001, n=4-5/group; FIG. 4B). In the GCL, Ki67-positive (Ki67+) cell densities were higher in Tat+ mice that had received BDNF-CT compared to saline treated Tat+ (P<0.0001) and Tat− mice (P<0.01). Notably, after 7 days of Tat induction, a decrease in Ki67 cell densities (P<0.05) was observed in Tat+ versus Tat− mice.

### 3) Assessment of Neurogenesis

BDNF-CT treatment increased neurogenesis (F_{(2,9)}=24.69, P=0.0002, n=4/group; FIG. 4C). In the GCL, doublecortin-positive (DCX+) cell densities were higher in Tat+ mice that had received BDNF-CT compared to saline-treated Tat+ (P<0.01) and Tat− mice (P<0.001; FIG. 4C). Representative images show BrdU+ (FIG. 4D-F), Ki67+ (FIG. 4G-I) and DCX+ (FIG. 4J-L) staining in the hippocampus.

### 4) Assessment of Newborn Cell Differentiation into Young Neurons

BDNF-CT treatment (FIG. 5A) enhanced differentiation of newborn cells into young neurons (F_{(2,6)}=43.66, P=0.0003, n=3/group; FIG. 5B). In the GCL, the mean percentage of double-labeled cells (BrdU+ and DCX+, FIG. 5I-K) was significantly higher in Tat+ mice that had received BDNF-CT compared to saline-treated Tat+ (P<0.001) and Tat− mice (P<0.05). After 7 days of Tat induction, a decrease in the mean percentage of double-labeled cells (BrdU+ and DCX+) (P<0.01) was found in Tat+ versus Tat− mice (FIG. 5B).

### Example 7. BDNF-CT Enhanced Synaptogenesis and Dendritic Integrity in the Hippocampal Regions

BDNF-CT administration (FIG. 6A) increased synaptogenesis (FIG. 6B) and dendritic integrity (FIG. 6C) in the hippocampus. The hippocampal synaptophysin (SYP) IR was higher in the DG (t-Test, n=6-8/per group, P=0.0007), CA1 (P=0.0177) and CA3 (P=0.0003) regions of Tat+mice that had received BDNF-CT compared to saline treated Tat+ controls (FIG. 6D). The hippocampal microtubule associated protein 2 (MAP2) IR also was increased in the DG (t-Test, N=6-8/per group, P=0.0052), CA1 (P=0.0195) and CA3 (P=0.0396) regions of Tat+ mice that had received BDNF-CT compared to saline-treated Tat+ controls (FIG. 6E).

### Example 8. BDNF-CT Enhanced Learning, Memory, and Cognitive Flexibility

### 1) Recognition Memory

There was an effect of treatment (F_{(2,17)}=4.138, P=0.0343, n=5-10/group; FIG. 7A) on recognition index (RI) of the Novel Object Recognition Test (NORT). Tat+ mice that had received BDNF-CT spent more time exploring the novel object. Post hoc analysis revealed dose-related effects. The % RI values were higher in animals that were given a high dose (0.3 mg/kg of BDNF & 2.4 mg/kg of CT; P<0.05), but not a low dose of BDNF-CT (0.15 mg/kg of BDNF & 1.2 mg/kg of CT; P>0.05), when compared to saline treated Tat+ controls. Representative heat maps show time spent in different locations of the NORT testing chamber (FIG. 7B).

### 2) Spatial Learning and Memory

During the acquisition phase (N =30; FIG. 7C) of the Barnes Maze Test (BMT), there was an overall effect of treatment (F_{(1,28.3)}=4.383, P=0.0454) and day (F_{(3,25.1)}=16.342, P<0.0001) on mean escape latency. Tat+ mice (n=14) given BDNF-CT spent less time locating and entering the escape chamber than saline-treated Tat+ controls (n=16) (FIG. 7E). No treatment group differences were found in numbers of errors (P>0.05, FIG. 7G). There was an overall effect of treatment (F_{(1,27.2)}=7.243, P=0.012) on average speed which increased in BDNF-CT vs. saline treated Tat+ mice (FIG. 11A). However, no treatment group differences were found in path efficiencies (P>0.05, FIG. 11B).

### 3) Cognitive Flexibility

In the BMT reversal learning phase (N=30; FIG. 7D), there were overall effects of treatment (F_{(1,32.1)}=11.102, P=0.0022) and trial (F_{(3,79.4)}=11.183, P<0.0001) on escape latency. Tat+ mice (n=14) given BDNF-CT performed better than saline treated Tat+ controls (n=16) (FIG. 7f). It was also found an overall effect of treatment (F_{(1,107)}=5.112, P=0.0258) and trial (F_{(3,107)}=3.693, P=0.0142) on numbers of reference errors (FIG. 7H). Overall effects of trial (F_{(3,24.5)}=5.459, P=0.0051) were found for average speed (FIG. 11C). It was also found overall effects of treatment (F_{(1,43.7)}=4.881, P=0.0325) and trial (F_{(3,75.8)}=3.053, P=0.0335) on path efficiency (FIG. 11D).

### 4) Correlations Between Cognitive Performance and Markers of Synaptogenesis and Dendritic Integrity

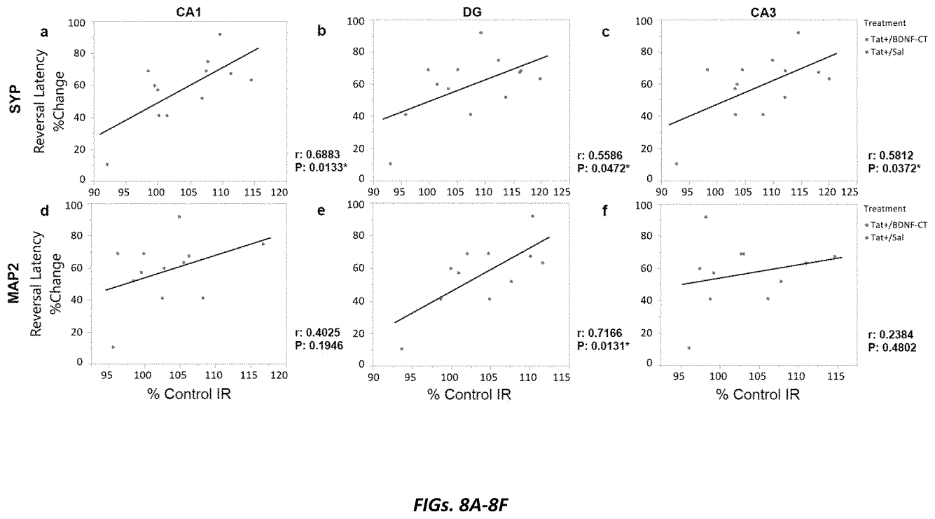

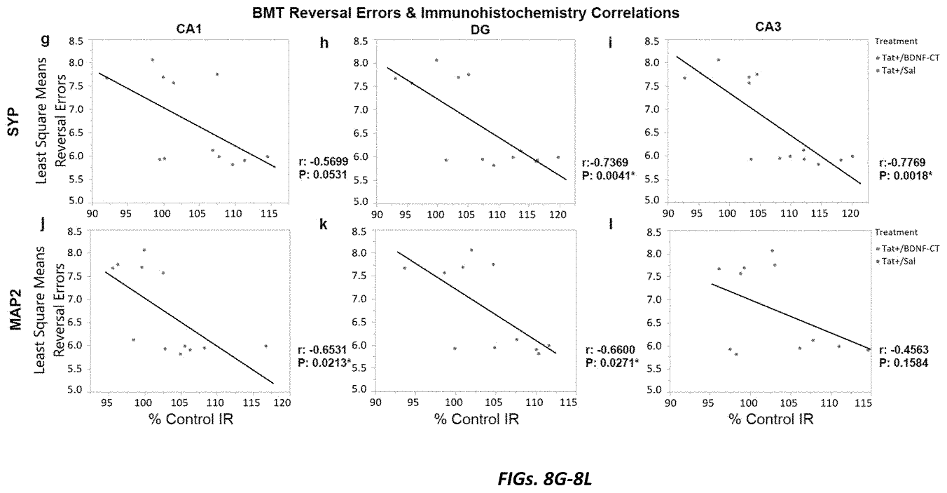

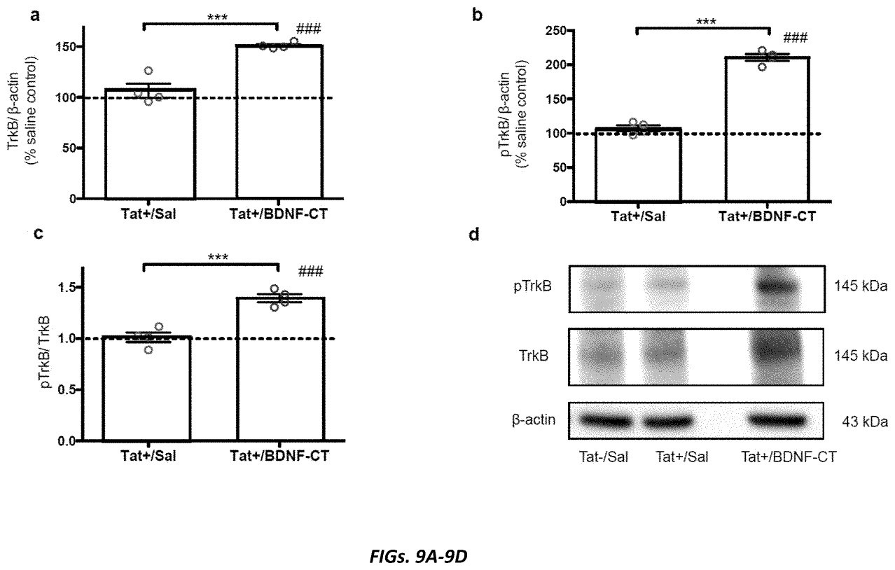

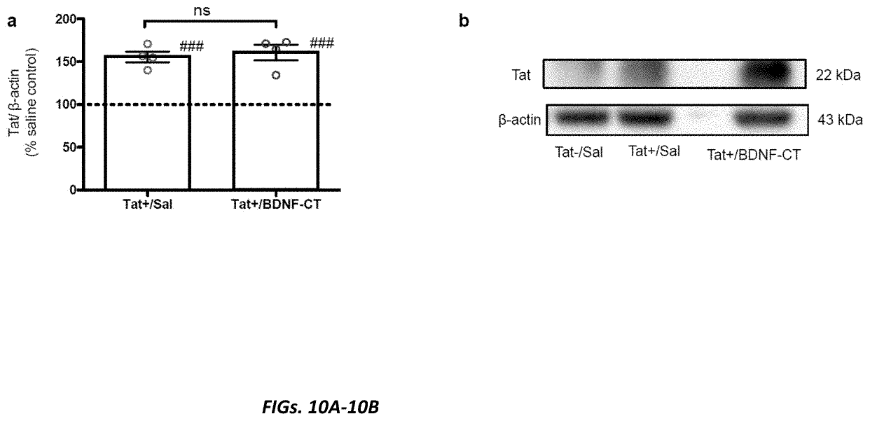

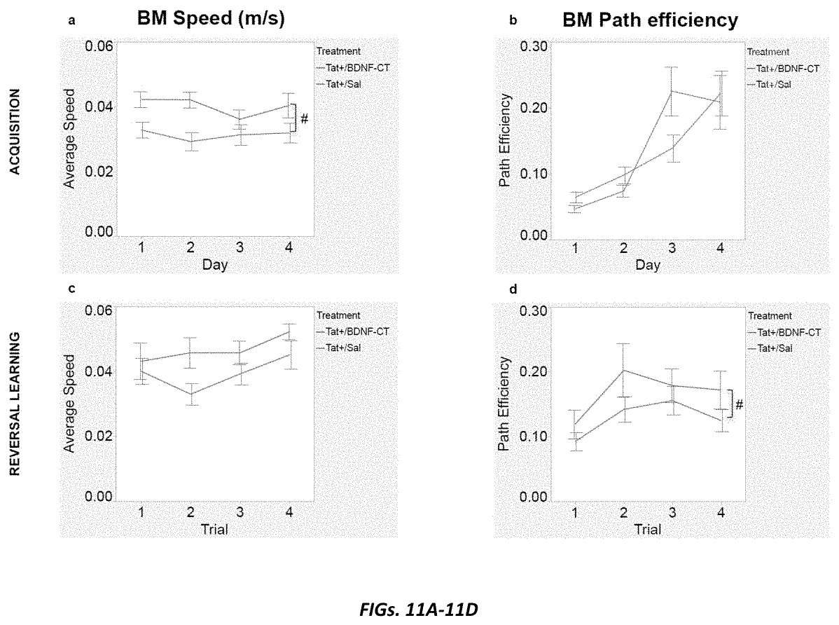

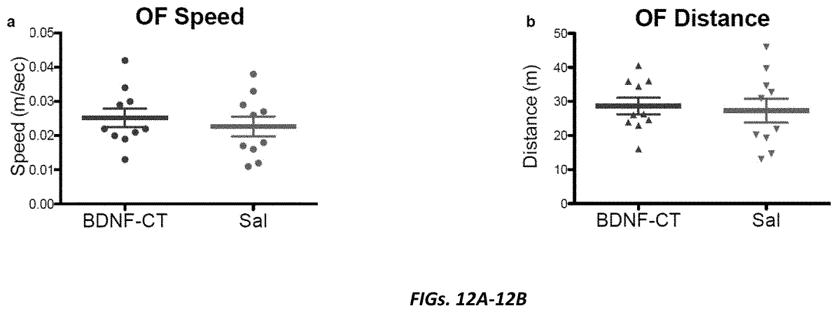

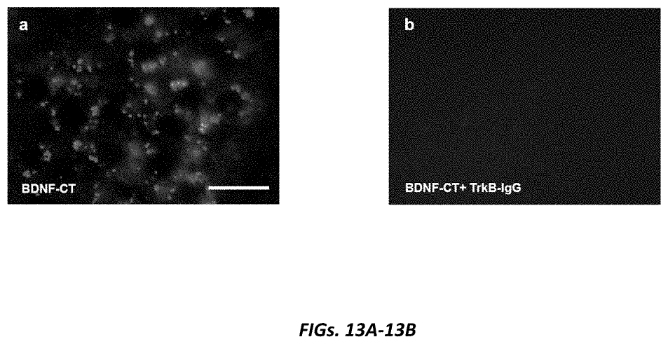

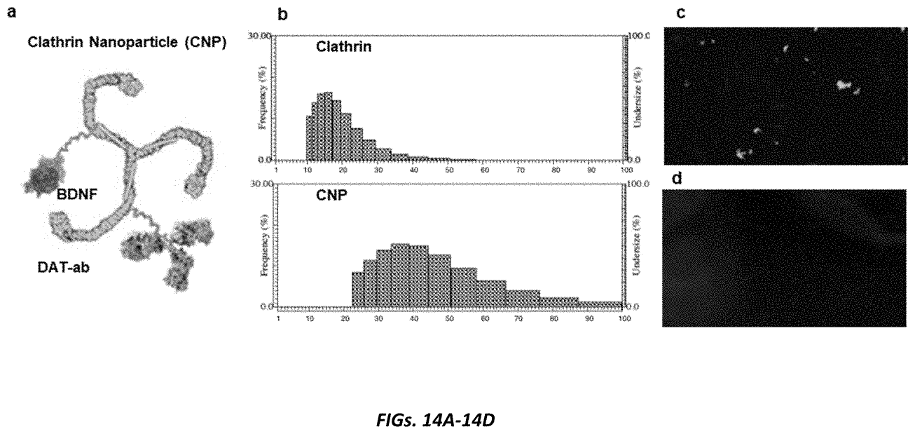

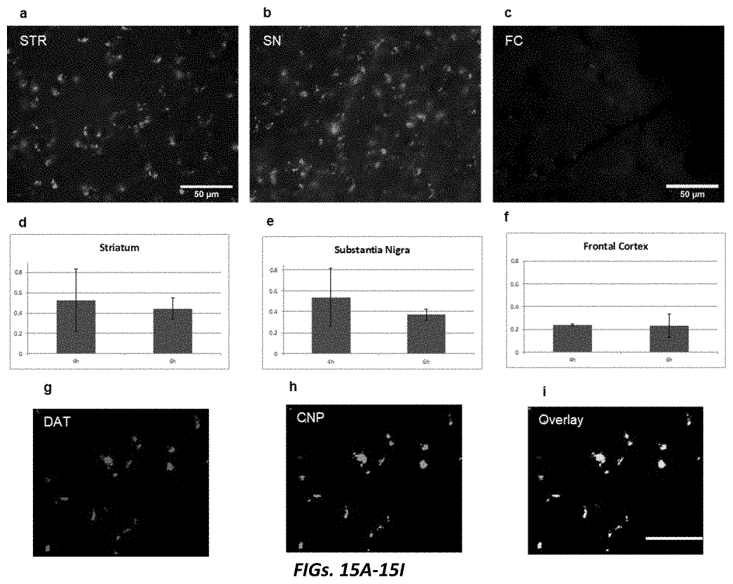

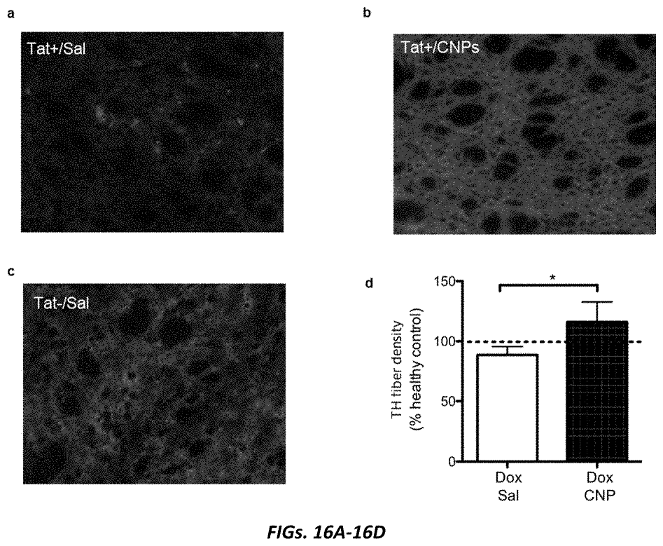

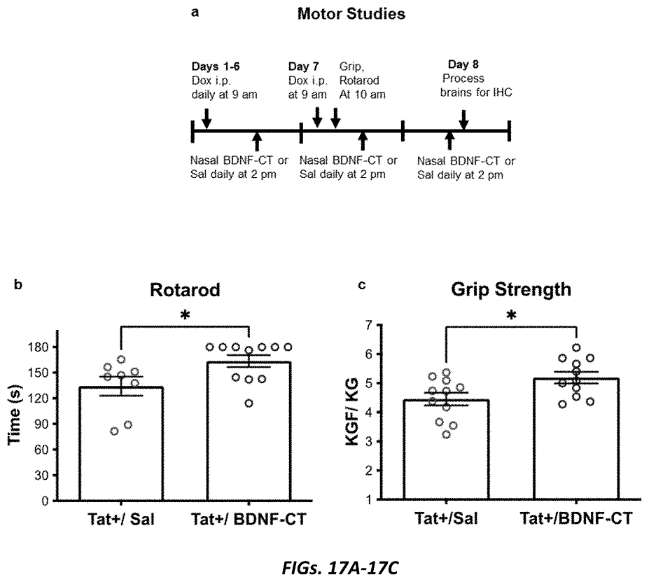

Correlations between BMT variables (% change in the latency to escape and reference errors) and hippocampal markers for synaptic density (SYP) and dendritic integrity (MAP2) were assessed in Tat+ mice treated with BDNF-CT or saline (FIG. 8). Significant correlations were found between improved reversal learning task performance (% change in the latency to escape) and increased SYP IR in the CA1 (r=0.6883, P=0.0133), DG (r=0.5586, P=0.0472) and CA3 (r=0.5812, P=0.0372) regions of the hippocampus (FIG. 8A-C). Similar relationships existed between improved reversal learning task performance and MAP2 IR in the DG region (r=0.7166, P=0.0131) (FIG. 8E).

During the reversal learning task, inverse relationships were found between performance errors (FIG. 8) and SYP IR in the DG (r=−0.7369, P=0.0041) and CA3 (r=−0.7769, P=0.0018) regions of the hippocampus. Also, there was a tendency for lower numbers of performance errors to correlate with enhanced SYP IR in the CA1 region (r=−0.5699, P=0.0531) (FIG. 8G). In addition, significant correlations were found between reversal learning task performance errors and MAP2 IR in the CA1 (r=−0.6531, P=0.0213) and DG (r=−0.6600, P=0.0271) regions of the hippocampus (FIG. 8J-K).

### Example 9. BDNF-CT Did not Exhibit Toxic Effects on Motor Functions in Healthy Mice

BDNF-CT treatment did not exhibit significant effects on average speed (FIG. 12A) and total distance traveled (FIG. 12B) in the Open Field (OF) Test. The average speed and distance traveled were not significantly different (P>0.05) in BDNF-CT vs. saline treated healthy C57BL/6J mice (FIG. 12).

### Example 10. Mice Motor Studies

Methods: Clathrin nanoparticles (CNPs) were synthesized by conjugating DAT-Ab and BDNF to Clathrin carrier using polyethylene glycols (PEGs) at 1:3:1 molar ratio per published protocol (Vitaliano at al 2022). Fluorescent and radioactive labeled CNPs were also produced and brain biodistribution of CNPs was tested according to published protocol (Vitaliano at al 2022). iTat mice (N=22) that model HIV-associated Neurocognitive Disorder (HAND) were used to test CNPs. Mice were treated once daily at 9 AM with saline or doxycycline (Dox, 100 mg/kg, i.p.) to induce neurotoxic Tat protein expression in the brain. At 2 PM, Dox treated (Tat+) mice received daily i.n. saline or CNPs (with 2.4 mg/kg or clathrin, 0.3 mg/kg BDNF and 0. 55 mg/kg of DAT-Abs). Rotarod and forelimb grip tests were performed on the 7^{th} day. Mice were sacrificed on the day 8^{th} of treatments administration. Their brains were perfused, fixed and cryoprotected. Slide mounted sections were incubated with anti-tyrosine hydroxylase (TH) primary mAbs (Santa Cruz 25269) and then with goat anti-mouse Alexa Fluor 568 secondary Abs, and evaluated using Leica TCS-NT confocal microscope, or a Zeiss Axio Scope A1 and NIH ImageJ software.

Results: Results for this Example are shown in FIGS. 14-17. Clathrin triskelia with anti-DAT-antibody (DATab) and BDNF were generated (see, FIG. 14). Clathrin triskelia with anti-DAT-antibody (DATab) and BDNF (0.3 mg/kg of BDNF, 0.55 mg/kg of DATab and 2.4 mg/kg of CT) specifically targeted DA brain regions after i.n delivery, and increased striatal BDNF concentrations over 100 times above that achieved previously with systemic delivery of BDNF (see, FIG. 15). Striatal tyrosine hydroxylase densities were higher (P=0.0467) in CNP vs. saline treated Tat+ mice (see, FIG. 16). CNP-treated Tat+ mice exhibited higher grip strength (P=0.0105) and improved rotarod performance (P=0.0156) compared to saline treated Tat+ mice (see, FIG. 17).

CNPs successfully bypassed the BBB and delivered adequate concentrations of BDNF to neurons expressing DAT in the mouse brain. NPs rescued striatal tyrosine hydroxylase-positive fibers from HIV/Tat neurotoxicity and improved motor strength and performance. Thus, targeted Clathrin-based NPs could assist in early treatment of dopaminergic neurodegeneration in neurodegenerative and neuroinflammatory disorders.

### REFERENCES

1. Illum, L. Nasal drug delivery—recent developments and future prospects. J Control Release 161, 254-263 (2012).
2. Gomez, D., Martinez, J. A., Hanson, L. R., Frey, W. H., 2nd & Toth, C. C. Intranasal treatment of neurodegenerative diseases and stroke. Frontiers in bioscience 4, 74-89 (2012).
3. Zhu, J., Jiang, Y., Xu, G. & Liu, X. Intranasal administration: a potential solution for BBB delivering neurotrophic factors. Histology and histopathology 27, 537-548 (2012).
4. Vitaliano, G. D., Vitaliano, F., Rios, J. D., Renshaw, P. F. & Teicher, M. H. New clathrin-based nanoplatforms for magnetic resonance imaging. PloS one 7, e35821 (2012).
5. Brodsky, F. M. Diversity of clathrin function: new tricks for an old protein. Annu Rev Cell Dev Biol 28, 309-336 (2012).
6. Kirchhausen, T., Owen, D. & Harrison, S. C. Molecular structure, function, and dynamics of clathrin-mediated membrane traffic. Cold Spring Harb Perspect Biot 6, a016725 (2014).
7. Preston, J. E., Joan Abbott, N. & Begley, D. J. Transcytosis of macromolecules at the blood-brain barrier. Advances in pharmacology 71, 147-163 (2014).
8. Granseth, B., Odermatt, B., Royle, S. J. & Lagnado, L. Clathrin-mediated endocytosis: the physiological mechanism of vesicle retrieval at hippocampal synapses. The Journal of physiology 585, 681-686 (2007).
9. Mills, I. G. The interplay between clathrin-coated vesicles and cell signalling. Seminars in cell & developmental biology 18, 459-470 (2007).
10. Royle, S. J. The cellular functions of clathrin. Cellular and molecular life sciences: CMLS 63, 1823-1832 (2006).
11. Baba, T., et al. Clathrin-dependent and clathrin-independent endocytosis are differentially sensitive to insertion of poly (ethylene glycol)-derivatized cholesterol in the plasma membrane. Traffic (Copenhagen, Denmark) 2, 501-512 (2001).
12. Schmid, S. L., Matsumoto, A. K. & Rothman, J. E. A domain of clathrin that forms coats. Proceedings of the National Academy of Sciences of the United States of America 79, 91-95 (1982).
13. Bartus, R. T. & Johnson, E. M., Jr. Clinical tests of neurotrophic factors for human neurodegenerative diseases, part 1: Where have we been and what have we learned? 848 Neurobiol Dis 97, 156-168 (2017).
14. Lu, B., Nagappan, G., Guan, X., Nathan, P. J . & Wren, P. BDNF-based synaptic repair as a disease-modifying strategy for neurodegenerative diseases. Nature reviews. Neuroscience 14, 401-416 (2013).
15. Ghosh, A., Carnahan, J. & Greenberg, M. E. Requirement for BDNF in activity-dependent survival of cortical neurons. Science 263, 1618-1623 (1994).
16. Horch, H. W. & Katz, L. C. BDNF release from single cells elicits local dendritic growth in nearby neurons. Nature neuroscience 5, 1177-1184 (2002).
17. Zagrebelsky, M. & Korte, M. Form follows function: BDNF and its involvement in sculpting the function and structure of synapses. Neuropharmacology 76 Pt C, 628-638 (2014).
18. Mattson, M. P., Maudsley, S. & Martin, B. BDNF and 5-HT: a dynamic duo in age-related neuronal plasticity and neurodegenerative disorders. Trends in neurosciences 27, 589-594 (2004).
19. Leal, G., Afonso, P. M., Salazar, I. L. & Duarte, C. B. Regulation of hippocampal synaptic plasticity by BDNF. Brain research 1621, 82-101 (2015).
20. Song, M., Martinowich, K. & Lee, F. S. BDNF at the synapse: why location matters. Molecular psychiatry 22, 1370-1375 (2017).
21. Yamada, K., Mizuno, M. & Nabeshima, T. Role for brain-derived neurotrophic factor in learning and memory. Life sciences 70, 735-744 (2002).
22. Cunha, C., Brambilla, R. & Thomas, K. L. A simple role for BDNF in learning and memory? Frontiers in molecular neuroscience 3, 1 (2010).
23. Bekinschtein, P., Cammarota, M. & Medina, J. H. BDNF and memory processing. Neuropharmacology 76 Pt C, 677-683 (2014).
24. Andero, R., Choi, D. C. & Ressler, K. J. BDNF-TrkB receptor regulation of distributed adult neural plasticity, memory formation, and psychiatric disorders. Progress in molecular biology and translational science 122, 169-192 (2014).
25. Nagahara, A. H., et al. Neuroprotective effects of brain-derived neurotrophic factor in rodent and primate models of Alzheimer's disease. Nat Med 15, 331-337 (2009).
26. Nagahara, A. H. & Tuszynski, M. H. Potential therapeutic uses of BDNF in neurological and psychiatric disorders. Nature reviews. Drug discovery 10, 209-219 (2011).
27. Poduslo, J. F. & Curran, G. L. Permeability at the blood-brain and blood-nerve barriers of the neurotrophic factors: NGF, CNTF, NT-3, BDNF. Brain research. Molecular brain research 36, 280-286 (1996).
28. Pardridge, W. M., Kang, Y. S. & Buciak, J. L. Transport of human recombinant brain-derived neurotrophic factor (BDNF) through the rat blood-brain barrier in vivo using vector-mediated peptide drug delivery. Pharmaceutical research 11, 738-746 (1994).
29. Alcala-Barraza, S. R., et al. Intranasal delivery of neurotrophic factors BDNF, CNTF, EPO, and NT-4 to the CNS. Journal of drug targeting 18, 179-190 (2010).
30. Scharfman, H., et al. Increased neurogenesis and the ectopic granule cells after intrahippocampal BDNF infusion in adult rats. Experimental neurology 192, 348-356 (2005).
31. Bekinschtein, P., et al. BDNF is essential to promote persistence of long-term memory storage. Proceedings of the National Academy of Sciences of the United States of America 105, 2711-2716 (2008).
32. Kuipers, S. D., et al. BDNF-induced LTP is associated with rapid Arc/Arg3.1-dependent enhancement in adult hippocampal neurogenesis. Scientific reports 6, 21222 (2016). 895
33. Blurton-Jones, M., et al. Neural stem cells improve cognition via BDNF in a transgenic model of Alzheimer disease. Proc Natl Acad Sci USA 106, 13594-13599 (2009).
34. Zhang, W., et al. Neural stem cell transplants improve cognitive function without altering amyloid pathology in an APP/PS1 double transgenic model of Alzheimer's disease. Mol Neurobiol 50, 423-437 (2014).
35. Ando, S., et al. Animal model of dementia induced by entorhinal synaptic damage and partial restoration of cognitive deficits by BDNF and carnitine. J Neurosci Res 70, 519-527 (2002).
36. Quesseveur, G., et al. BDNF overexpression in mouse hippocampal astrocytes promotes local neurogenesis and elicits anxiolytic-like activities. Translational psychiatry 3, e253 (2013).
37. Sirianni, R. W., Olausson, P., Chiu, A. S., Taylor, J. R. & Saltzman, W. M. The behavioral and biochemical effects of BDNF containing polymers implanted in the hippocampus of rats. Brain research 1321, 40-50 (2010).
38. Angelov, B., et al. Multicompartment lipid cubic nanoparticles with high protein upload: millisecond dynamics of formation. ACS Nano 8, 5216-5226 (2014).
39. Chen, H., et al. Focused ultrasound-enhanced intranasal brain delivery of brain-derived neurotrophic factor. Scientific reports 6, 28599 (2016).
40. Geral, C., Angelova, A. & Lesieur, S. From molecular to nanotechnology strategies for delivery of neurotrophins: emphasis on brain-derived neurotrophic factor (BDNF). Pharmaceutics 5, 127-167 (2013).
41. Zhang, Y. & Pardridge, W. M. Blood-brain barrier targeting of BDNF improves motor function in rats with middle cerebral artery occlusion. Brain research 1111, 227-229 (2006).
42. Khalin, I., et al. Brain-derived neurotrophic factor delivered to the brain using poly (lactide-co-glycolide) nanoparticles improves neurological and cognitive outcome in mice with traumatic brain injury. Drug delivery 23, 3520-3528 (2016).
43. Jiang, Y., et al. Nanoformulation of Brain-Derived Neurotrophic Factor with Target Receptor-Triggered-Release in the Central Nervous System. Advanced functional materials 28(2018).
44. Ma, X. C., et al. Intranasal Delivery of Recombinant AAV Containing BDNF Fused with HA2TAT: a Potential Promising Therapy Strategy for Major Depressive Disorder. Scientific reports 6, 22404 (2016).
45. Kim, B. O., et al. Neuropathologies in transgenic mice expressing human immunodeficiency virus type 1 Tat protein under the regulation of the astrocyte-specific glial fibrillary acidic protein promoter and doxycycline. The American journal of pathology 162, 1693-1707 (2003).
46. Langford, D., et al. Doxycycline-inducible and astrocyte-specific HIV-1 Tat transgenic mice (iTat) as an HIV/neuroAIDS model. Journal of neurovirology 24, 168-179 (2018). 934
47. Mocchetti, I., Bachis, A., Campbell, L. A. & Avdoshina, V. Implementing neuronal plasticity in NeuroAIDS: the experience of brain-derived neurotrophic factor and other neurotrophic factors. Journal of neuroimmune pharmacology: the official journal of the Society on Neurolmmune Pharmacology 9, 80-91 (2014).
48. Bachis, A., Avdoshina, V., Zecca, L., Parsadanian, M. & Mocchetti, I. Human immunodeficiency virus type 1 alters brain-derived neurotrophic factor processing in neurons. The Journal of neuroscience: the official journal of the Society for Neuroscience 32, 9477-9484 (2012).
49. Albrecht, D., et al. Trophic factors in cerebrospinal fluid and spinal cord of patients with tropical spastic paraparesis, HIV, and Creutzfeldt-Jakob disease. AIDS research and human retroviruses 22, 248-254 (2006).
50. Meeker, R. B., Poulton, W., Markovic-Plese, S., Hall, C. & Robertson, K. Protein changes in CSF of HIV-infected patients: evidence for loss of neuroprotection. Journal of neurovirology 17, 258-273 (2011).
51. Fields, J., et al. HIV-1 Tat alters neuronal autophagy by modulating autophagosome fusion to the lysosome: implications for HIV-associated neurocognitive disorders. The Journal of neuroscience: the official journal of the Society for Neuroscience 35, 1921-1938 (2015).
52. King, J. E., Eugenin, E. A., Buckner, C. M. & Berman, J. W. HIV tat and neurotoxicity. Microbes and infection/Institut Pasteur 8, 1347-1357 (2006).
53. Hudson, L., et al. Detection of the human immunodeficiency virus regulatory protein tat in CNS tissues. Journal of neurovirology 6, 145-155 (2000).
54. Henderson, L. J., et al. Presence of Tat and transactivation response element in spinal fluid despite antiretroviral therapy. Aids 33 Suppl 2, S145-S157 (2019).
55. Jones, M., Olafson, K., Del Bigio, M. R., Peeling, J. & Nath, A. Intraventricular injection of human immunodeficiency virus type 1 (HIV-1) tat protein causes inflammation, gliosis, apoptosis, and ventricular enlargement. Journal of neuropathology and experimental neurology 57, 563-570 (1998).
56. McLaughlin, J. P., et al. Conditional Human Immunodeficiency Virus Transactivator of Transcription Protein Expression Induces Depression-like Effects and Oxidative Stress. Biological psychiatry. Cognitive neuroscience and neuroimaging 2, 599-609 (2017).
57. Carey, A. N., et al. Conditional Tat protein expression in the GT-tg bigenic mouse brain induces gray matter density reductions. Progress in neuro-psychopharmacology & biological psychiatry 43, 49-54 (2013).
58. Rahimian, P. & He, J. J. HIV-1 Tat-shortened neurite outgrowth through regulation of microRNA-132 and its target gene expression. Journal of neuroinflammation 13, 247 970 (2016).
59. Carey, A. N., Sypek, E. I., Singh, H. D., Kaufman, M. J. & McLaughlin, J. P. Expression of HIV- Tat protein is associated with learning and memory deficits in the mouse. Behavioural brain research 229, 48-56 (2012).
60. Marks, W. D., et al. HIV-1 Tat causes cognitive deficits and selective loss of parvalbumin, somatostatin, and neuronal nitric oxide synthase expressing hippocampal CA1 interneuron subpopulations. Journal of neurovirology 22, 747-762 (2016).
61. Fitting, S., et al. Synaptic dysfunction in the hippocampus accompanies learning and memory deficits in human immunodeficiency virus type-1 Tat transgenic mice. Biological psychiatry 73, 443-453 (2013).
62. Ramirez, S. H., et al. Neurotrophins prevent HIV Tat-induced neuronal apoptosis via a nuclear factor-kappaB (NF-kappaB)-dependent mechanism. Journal of neurochemistry 78, 874-889 (2001).
63. Fujimura, R. K., et al. HIV-1 proviral DNA load across neuroanatomic regions of individuals with evidence for HIV-1-associated dementia. Journal of acquired immune deficiency syndromes and human retrovirology: official publication of the International Retrovirology Association 16, 146-152 (1997).
64. Wiley, C. A., et al. Distribution of brain HIV load in AIDS. Brain pathology 8, 277-284 (1998).
65. Anthony, I. C., Ramage, S. N., Carnie, F. W., Simmonds, P. & Bell, J. E. Influence of HAART on HIV-related CNS disease and neuroinflammation. Journal of neuropathology and experimental neurology 64, 529-536 (2005).
66. Nir, T. M., et al. Association of Immunosuppression and Viral Load with Subcortical Brain Volume in an International Sample of People Living With HIV. JAMA Netw Open 4, e2031190 (2021).
67. Ferguson, M. L., et al. Conformation of a clathrin triskelion in solution. Biochemistry 45, 5916-5922 (2006).
68. Kocsis, E., Trus, B. L., Steer, C. J., Bisher, M. E. & Steven, A. C. Image averaging of flexible fibrous macromolecules: the clathrin triskelion has an elastic proximal segment. Journal of structural biology 107, 6-14 (1991).
69. Kirchhausen, T., Harrison, S. C. & Heuser, J. Configuration of clathrin trimers: evidence from electron microscopy. Journal of ultrastructure and molecular structure research 94, 199-208 (1986).
70. Kotova, S., et al. AFM visualization of clathrin triskelia under fluid and in air. FEBS letters 584, 44-48.
71. Fitting, S., et al. Interactive comorbidity between opioid drug abuse and HIV-1 Tat: chronic exposure augments spine loss and sublethal dendritic pathology in striatal neurons. The American journal of pathology 177, 1397-1410 (2010).
72. Lee, R., Kermani, P., Teng, K. K. & Hempstead, B. L. Regulation of cell survival by secreted proneurotrophins. Science 294, 1945-1948 (2001).
73. Yasuda, M., et al. Robust stimulation of TrkB induces delayed increases in BDNF and Arc mRNA expressions in cultured rat cortical neurons via distinct mechanisms. Journal of neurochemistry 103, 626-636 (2007).
74. Yang, J., et al. proBDNF negatively regulates neuronal remodeling, synaptic transmission, and synaptic plasticity in hippocampus. Cell reports 7, 796-806 (2014).
75. Guo, W., Nagappan, G. & Lu, B. Differential effects of transient and sustained activation of BDNF-TrkB signaling. Developmental neurobiology 78, 647-659 (2018).
76. Panja, D. & Bramham, C. R. BDNF mechanisms in late LTP formation: A synthesis and breakdown. Neuropharmacology 76 Pt C, 664-676 (2014).
77. Patterson, S. L., et al. Recombinant BDNF rescues deficits in basal synaptic transmission and hippocampal LTP in BDNF knockout mice. Neuron 16, 1137-1145 (1996).
78. Kang, H. & Schuman, E. M. Long-lasting neurotrophin-induced enhancement of synaptic transmission in the adult hippocampus. Science 267, 1658-1662 (1995).
79. Rossi, C., et al. Brain-derived neurotrophic factor (BDNF) is required for the enhancement of hippocampal neurogenesis following environmental enrichment. The European journal of neuroscience 24, 1850-1856 (2006).
80. Woo, N. H., et al. Activation of p75NTR by proBDNF facilitates hippocampal long-term depression. Nature neuroscience 8, 1069-1077 (2005).
81. Teng, H. K., et al. ProBDNF induces neuronal apoptosis via activation of a receptor complex of p75NTR and sortilin. The Journal of neuroscience: the official journal of the Society for Neuroscience 25, 5455-5463 (2005).
82. Barnes, P. & Thomas, K. L. Proteolysis of proBDNF is a key regulator in the formation of memory. PloS one 3, e3248 (2008).
83. Qiao, H., An, S.C., Xu, C. & Ma, X. M. Role of proBDNF and BDNF in dendritic spine plasticity and depressive-like behaviors induced by an animal model of depression. Brain research 1663, 29-37 (2017).
84. Numakawa, T., et al. BDNF function and intracellular signaling in neurons. Histology and histopathology 25, 237-258 (2010).
85. Brunet, A., Datta, S. R. & Greenberg, M. E. Transcription-dependent and -independent control of neuronal survival by the PI3K-Akt signaling pathway. Current opinion in neurobiology 11, 297-305 (2001).
86. Yang, J. W., et al. BDNF promotes the growth of human neurons through crosstalk with the Wnt/beta-catenin signaling pathway via GSK-3beta. Neuropeptides 54, 35-46 (2015).
87. Green, M. V. & Thayer, S. A. NMDARs Adapt to Neurotoxic HIV Protein Tat Downstream of a GluN2A-Ubiquitin Ligase Signaling Pathway. The Journal of neuroscience: the official journal of the Society for Neuroscience 36, 12640-12649 (2016).
88. Fassnacht, M., et al. AKT is highly phosphorylated in pheochromocytomas but not in benign adrenocortical tumors. J Clin Endocrinol Metab 90, 4366-4370 (2005).
89. Yan, Q., et al. Expression of brain-derived neurotrophic factor protein in the adult rat central nervous system. Neuroscience 78, 431-448 (1997).
90. Barde, Y. A., Edgar, D. & Thoenen, H. Purification of a new neurotrophic factor from mammalian brain. The EMBO journal 1, 549-553 (1982).
91. Lindholm, D., Carroll, P., Tzimagiorgis, G. & Thoenen, H. Autocrine-paracrine regulation of hippocampal neuron survival by IGF-1 and the neurotrophins BDNF, NT-3 and NT-4. The European journal of neuroscience 8, 1452-1460 (1996).
92. Johnson-Farley, N. N., Travkina, T. & Cowen, D. S. Cumulative activation of akt and consequent inhibition of glycogen synthase kinase-3 by brain-derived neurotrophic factor and insulin-like growth factor-1 in cultured hippocampal neurons. The Journal of pharmacology and experimental therapeutics 316, 1062-1069 (2006).
93. Vaka, S. R., Murthy, S. N., Balaji, A. & Repka, M. A. Delivery of brain-derived neurotrophic factor via nose-to-brain pathway. Pharm Res 29, 441-447 (2012).
94. Zheng, F., Soellner, D., Nunez, J. & Wang, H. The basal level of intracellular calcium gates the activation of phosphoinositide 3-kinase-Akt signaling by brain-derived neurotrophic factor in cortical neurons. Journal of neurochemistry 106, 1259-1274 (2008).
95. Numakawa, T., Odaka, H. & Adachi, N. Actions of Brain-Derived Neurotrophin Factor in the Neurogenesis and Neuronal Function, and Its Involvement in the Pathophysiology of Brain Diseases. International journal of molecular sciences 19(2018).
96. Lian, D., et al. Exogenous BDNF increases neurogenesis in the hippocampus in experimental Streptococcus pneumoniae meningitis. J Neuroimmunol 294, 46-55 (2016).
97. Fatima, M., et al. Tripartite containing motif 32 modulates proliferation of human neural precursor cells in HIV-1 neurodegeneration. Cell death and differentiation 23, 776-786 (2016).
98. Mishra, M., Taneja, M., Malik, S., Khalique, H. & Seth, P. Human immunodeficiency virus type 1 Tat modulates proliferation and differentiation of human neural precursor cells: implication in NeuroAIDS. Journal of neurovirology 16, 355-367 (2010).
99. Fan, Y., Gao, X., Chen, J., Liu, Y. & He, J. J. HIV Tat Impairs Neurogenesis through Functioning As a Notch Ligand and Activation of Notch Signaling Pathway. The Journal of neuroscience: the official journal of the Society for Neuroscience 36, 11362-11373 (2016).
100. Hill, J. D., Zuluaga-Ramirez, V., Gajghate, S., Winfield, M. & Persidsky, Y. Chronic Intrahippocampal Infusion of HIV-1 Neurotoxic Proteins: A Novel Mouse Model of HIV-1 Associated Inflammation and Neural Stem Cell Dysfunction. Journal of neuroimmune pharmacology: the official journal of the Society on Neurolmmune Pharmacology 14, 375-382 (2019).
101. Coffey, E. T., Akerman, K. E. & Courtney, M. J. Brain derived neurotrophic factor induces a rapid upregulation of synaptophysin and tau proteins via the neurotrophin receptor TrkB in rat cerebellar granule cells. Neuroscience letters 227, 177-180 (1997).
102. Bamji, S. X., Rico, B., Kimes, N. & Reichardt, L. F. BDNF mobilizes synaptic vesicles and enhances synapse formation by disrupting cadherin-beta-catenin interactions. The Journal of cell biology 174, 289-299 (2006).
103. Fukumitsu, H., Ohashi, A., Nitta, A., Nomoto, H. & Furukawa, S. BDNF and NT-3 modulate expression and threonine phosphorylation of microtubule-associated protein 2 analogues, and alter their distribution in the developing rat cerebral cortex. Neuroscience letters 238, 107-110 (1997).
104. Melo, C. V., et al. Spatiotemporal resolution of BDNF neuroprotection against glutamate excitotoxicity in cultured hippocampal neurons. Neuroscience 237, 66-86 (2013).
105. Levine, A. J., et al. Multilevel analysis of neuropathogenesis of neurocognitive impairment in HIV. Journal of neurovirology 22, 431-441 (2016).
106. Shin, A. H. & Thayer, S. A. Human immunodeficiency virus-1 protein Tat induces excitotoxic loss of presynaptic terminals in hippocampal cultures. Molecular and cellular neurosciences 54, 22-29 (2013).
107. Butler, T. R., Smith, K. J., Self, R. L., Braden, B. B. & Prendergast, M. A. Neurodegenerative effects of recombinant HIV-1 Tat(1-86) are associated with inhibition of microtubule formation and oxidative stress-related reductions in microtubule-associated protein-2(a,b). Neurochemical research 36, 819-828 (2011).
108. Aprea, S., et al. Tubulin-mediated binding of human immunodeficiency virus-1 Tat to the cytoskeleton causes proteasomal-dependent degradation of microtubule-associated protein 2 and neuronal damage. The Journal of neuroscience: the official journal of the Society for Neuroscience 26, 4054-4062 (2006).
109. Maragos, W. F., et al. Neuronal injury in hippocampus with human immunodeficiency virus transactivating protein, Tat. Neuroscience 117, 43-53 (2003).

110. LaBel, C. P. & Foss, J. Use of a rodent neurotoxicity screening battery in the preclinical safety assessment of recombinant-methionyl human brain-derived neurotrophic factor. Neurotoxicology., 851-864 (1996).
111. Zhang, L., et al. Brain-derived neurotrophic factor ameliorates learning deficits in a rat model of Alzheimer's disease induced by abeta1-42. PLoS One 10, e0122415 (2015).
112. Mizuno, M., et al. Phosphatidylinositol 3-kinase: a molecule mediating BDNF-dependent spatial memory formation. Molecular psychiatry 8, 217-224 (2003).
113. Schmitt, U., Tanimoto, N., Seeliger, M., Schaeffel, F. & Leube, R. E. Detection of behavioral alterations and learning deficits in mice lacking synaptophysin. Neuroscience 162, 234-243 (2009).
114. Khuchua, Z., et al. Deletion of the N-terminus of murine map2 by gene targeting disrupts hippocampal cal neuron architecture and alters contextual memory. Neuroscience 119, 101-111 (2003).
115. Dickens, A. M., et al. Chronic low-level expression of HIV-1 Tat promotes a neurodegenerative phenotype with aging. Sci Rep 7, 7748 (2017).
116. Jaeger, L. B. & Nath, A. Modeling HIV-associated neurocognitive disorders in mice: new approaches in the changing face of HIV neuropathogenesis. Disease models & mechanisms 5, 313-322 (2012).
117. Paterson, R. W., et al. The emerging spectrum of COVID-19 neurology: clinical, radiological and laboratory findings. Brain: a journal of neurology (2020).
118. Zhu, Y., Drake, M. T. & Kornfeld, S. Adaptor protein 1-dependent clathrin coat assembly on synthetic liposomes and Golgi membranes. Methods in enzymology 329, 379-387 (2001).
119. Soderquist, R. G., et al. PEGylation of brain-derived neurotrophic factor for preserved biological activity and enhanced spinal cord distribution. Journal of biomedical materials research. Part A 91, 719-729 (2009).
120. Sakane, T. & Pardridge, W. M. Carboxyl-directed pegylation of brain-derived neurotrophic factor markedly reduces systemic clearance with minimal loss of biologic activity. Pharmaceutical research 14, 1085-1091 (1997).
121. Hahn, Y. K., et al. Effects of chronic HIV-1 Tat exposure in the CNS: heightened vulnerability of males versus females to changes in cell numbers, synaptic integrity, and behavior. Brain structure & function (2013).
122. Boado, R. J., Zhang, Y., Zhang, Y. & Pardridge, W. M. Genetic engineering, expression, and activity of a fusion protein of a human neurotrophin and a molecular Trojan horse for delivery across the human blood-brain barrier. Biotechnology and bioengineering 97, 1376-1386 (2007).
123. Kummer, U. Tritium radiolabeling of antibodies to high specific activity with N-succinimidyl [2,3-3H]propionate: use in detecting and analyzing monoclonal antibodies. Methods in enzymology 121, 670-678 (1986).
124. Chartoff, E. H., Mague, S. D., Barhight, M. F., Smith, A. M. & Carlezon, W. A., Jr. Behavioral and molecular effects of dopamine D1 receptor stimulation during naloxone-precipitated morphine withdrawal. The Journal of neuroscience: the official journal of the Society for Neuroscience 26, 6450-6457 (2006).
125. Dingwall, C., et al. Human immunodeficiency virus 1 tat protein binds trans-activation- responsive region (TAR) RNA in vitro. Proceedings of the National Academy of Sciences of the United States of America 86, 6925-6929 (1989).
126. Snyder, J. S., et al. Adult-born hippocampal neurons are more numerous, faster maturing, and more involved in behavior in rats than in mice. The Journal of neuroscience: the official journal of the Society for Neuroscience 29, 14484-14495 (2009).
127. Wojtowicz, J. M. & Kee, N. BrdU assay for neurogenesis in rodents. Nature protocols 1, 1399-1405 (2006).
128. Scholzen, T. & Gerdes, J. The Ki-67 protein: from the known and the unknown. Journal of cellular physiology 182, 311-322 (2000).
129. Couillard-Despres, S., et al. Doublecortin expression levels in adult brain reflect neurogenesis. The European journal of neuroscience 21, 1-14 (2005).
130. Knaus, P., Betz, H. & Rehm, H. Expression of synaptophysin during postnatal development of the mouse brain. Journal of neurochemistry 47, 1302-1304 (1986).
131. Caceres, A., Banker, G. A. & Binder, L. Immunocytochemical localization of tubulin and microtubule-associated protein 2 during the development of hippocampal neurons in culture. The Journal of neuroscience: the official journal of the Society for Neuroscience 6, 714-722 (1986).
132. Antunes, M. & Biala, G. The novel object recognition memory: neurobiology, test procedure, and its modifications. Cognitive processing 13, 93-110 (2012).
133. Akaike, H., Parzen, E., Tanabe, K. & Kitagawa, G. Selected papers of Hirotugu kaike, (Springer, New York, 1998).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising: (i) a CNS targeting agent; and (ii) a neurotrophic factor, wherein the CNS targeting agent and the neurotrophic factor are linked to a clathrin nanoparticle.

2. The composition of claim 1, wherein the clathrin nanoparticle comprises a clathrin cage.

3. The composition of claim 1, wherein the clathrin nanoparticle consists of a clathrin triskelion.

4. The composition of claim 1, wherein the clathrin nanoparticle comprises a complex consisting of 1 to 3 clathrin heavy chains.

5. The composition of claim 1, wherein the neurotrophic factor is a neurotrophic factor that binds to tropomyosin-related kinase receptor, optionally TrkA, TrkB, and/or TrkC, NT-3 receptor, NT-4 receptor, NT-5 receptor, neurturin receptor, persephin receptor, artemin receptor, ciliary neurotrophic factor receptor, p75 neurotrophin receptor, or leukemia inhibitory factor receptor.

6. The composition of claim 1, wherein the neurotrophic factor is linked to the clathrin nanoparticles by conjugation.

7. The composition of claim 6, wherein the neurotrophic factor is conjugated to the clathrin nanoparticles via PEG.

8. The composition of claim 4, wherein a clathrin heavy chain of the clathrin triskelion is linked to 1 to 5 molecules of the neurotrophic factor.

9. The composition of claim 4, wherein the clathrin nanoparticle comprises a complex consisting of 3 clathrin heavy chains.

10. The composition of claim 4, wherein each of the clathrin heavy chains is linked to 1 to 5 molecules of the neurotrophic factor.

11. The composition of claim 9, wherein each of the clathrin heavy chains is linked to 1 molecule of the neurotrophic factor.

12. The composition of claim 1, wherein the neurotrophic factor is NT-3, NT-4, NT-5, NT-6, neurturin, persephin, artemin, ciliary neurotrophic factor, nerve growth factor (NGF), or leukemia inhibitory factor.

13. The composition of claim 1, wherein the CNS targeting agent is an antibody.

14. The composition of claim 13, wherein the antibody comprises an anti-CD11b antibody, an anti-TrkB receptor antibody, an anti-DAT antibody, an anti-GABA antibody, an anti-SYP antibody, an anti-serotonin transporter antibody, an anti-dopamine-1 antibody, an anti-dopamine-2 antibody, an anti-dopamine-3 antibody, an anti-TREM2-antibody, an IL-6R-antibody, or an anti-TNF-α-antibody.

15. The composition of claim 14, wherein the antibody is a monoclonal antibody.

16. The composition of claim 1, wherein the CNS targeting agent is a ligand.

17. The composition of claim 16, wherein the ligand comprises a DAT ligand, a TSPO ligand, a GABA ligand, a serotonin transporter ligand, a D1 ligand, a D2 ligand, or a D3 ligand.

18. The composition of claim 1, further comprising an imaging contrast agent.

19. The composition of claim 18, wherein the imaging contrast agent comprises a gadolinium (Gd) based contrast agent, manganese-based, superparamagnetic iron oxide (SPIO), monocrystalline iron oxide nanoparticle (MION), cross-linked iron oxide (CLIO), or magneto-dendrimer contrast agent.

20. The composition of claim 1, further comprising one or more additional therapeutic agent.

21. The composition of claim 21, wherein the additional therapeutic agent comprises aripiprazole, baclofen, bupropion, d-AMP and MPH-SR, dextroamphetamine, gabapentin, ibudilast, methylphenidate, mirtazapine, modafinil, NAC, naltrexone, rivastigmine, or topiramate.

22. The composition of claim 1, further comprising, the neurotrophic agent, targeting agent, imaging contrast agent and/or additional therapeutic agent linked together with the clathrin nanoparticle.

23. A method for treating a human subject having or at risk for developing a neurodegenerative disorder, optionally Alzheimer's disease, HIV- Associated Neurocognitive Disorder (HAND), Parkinson's disease, or Huntington's diseases; a neuroinflammatory disease, optionally amyotrophic lateral sclerosis or multiple sclerosis; stroke; a neurological disorder associated with COVID-19, optionally a cognitive deficit; traumatic brain injury; or a psychiatric disorder, optionally depression, comprising administering to the human subject a composition comprising: (i) a CNS targeting agent; and (ii) a neurotrophic factor, wherein the CNS targeting agent and the neurotrophic factor are linked to a clathrin nanoparticle.

24. The method of claim 23, wherein the clathrin nanoparticle comprises a clathrin cage.

25. The method of claim 23, wherein the clathrin nanoparticle consists of a clathrin triskelion.

26. The method of claim 24, wherein the clathrin nanoparticle comprises a complex consisting of 1 to 3 clathrin heavy chains.

27. The method of claim 23, wherein the neurotrophic factor is a neurotrophic factor that binds to tropomyosin-related kinase receptor, optionally TrkA, TrkB, and/or TrkC; NT-3 receptor; NT-4 receptor; NT-5 receptor; neurturin receptor; persephin receptor; artemin receptor; ciliary neurotrophic factor receptor; p75 neurotrophin receptor; or leukemia inhibitory factor receptor.

28. The method of claim 23, wherein the neurotrophic factor is linked to the clathrin nanoparticles by conjugation.

29. The method of claim 28, wherein the neurotrophic factor is conjugated to the clathrin nanoparticles via PEG.

30. The method of claim 26, wherein a clathrin heavy chain of the clathrin triskelion is linked to 1 to 5 molecules of the neurotrophic factor.

31. The method of claim 26, wherein the clathrin nanoparticle comprises a complex consisting of 3 clathrin heavy chains.

32. The method of claim 26, wherein each of the clathrin heavy chains is linked to 1 to 5 molecules of the neurotrophic factor.

33. The method of claim 31, wherein each of the clathrin heavy chains is linked to 1 molecule of the neurotrophic factor.

34. The method of claim 23, wherein the neurotrophic factor is NT-3, NT-4, NT-5, NT-6, neurturin, persephin, artemin, ciliary neurotrophic factor, nerve growth factor (NGF), or leukemia inhibitory factor.

35. The method of claim 23, wherein the CNS targeting agent is an antibody.

36. The method of claim 35, wherein the antibody comprises wherein the antibody comprises an anti-CD11b antibody, an anti-TrkB receptor antibody, an anti-DAT antibody, an anti-GABA antibody, an anti-SYP antibody, an anti-serotonin transporter antibody, an anti-dopamine-1 antibody, an anti-dopamine-2 antibody, an anti-dopamine-3 antibody, an anti-TREM2-antibody, an IL-6R-antibody, or an anti-TNF-α-antibody.

37. The method of claim 36, wherein the antibody is a monoclonal antibody.

38. The method of claim 23, wherein the CNS targeting agent is a ligand.

39. The method of claim 38, wherein the ligand comprises a DAT ligand, a TSPO ligand, a GABA ligand, a serotonin transporter ligand, a D1 ligand, a D2 ligand, or a D3 ligand.

40. The method of claim 23, wherein the clathrin nanoparticle further comprises an imaging contrast agent.

41. The method of claim 40, wherein the imaging contrast agent comprises a gadolinium (Gd) based contrast agent, manganese-based, superparamagnetic iron oxide (SPIO), monocrystalline iron oxide nanoparticle (MION), cross-linked iron oxide (CLIO), or magneto-dendrimer contrast agent.

42. The method of claim 23, wherein the HIV-Associated Neurocognitive Disorder is asymptomatic neurocognitive impairment (ANI), mild neurocognitive disorder (MND), or HIV associated dementia (HAD).

43. The method of claim 23, further comprising one or more additional therapeutic agent.

44. The method of claim 43, wherein the additional therapeutic agent comprises aripiprazole, baclofen, bupropion, d-AMP, MPH-SR, dextroamphetamine, gabapentin, ibudilast, methylphenidate, mirtazapine, modafinil, NAC, naltrexone, rivastigmine, or topiramate.

45. The method of claim 23, further comprising, the neurotrophic agent, targeting agent, imaging contrast agent and/or additional therapeutic agent linked together with the clathrin nanoparticle.

46. The method of claim 23, further comprising imaging a region of the brain using the contrast agent during a course of treating the neurodegenerative disorder.

47. The method of claim 23, wherein the composition is delivered intranasally or intravenously.
